(19) United States
(12) Patent Application Publication
(10) Pub. No.: US 2002/0086331 A1
(43) Pub. Date: Jul. 4, 2002

(54) Crystal structure of worm NitFhit reveals that a Nit tetramer binds two Fhit dimers

(76) Inventors: Carlo Croce, Philadelphia, PA (US); Charles Brenner, Philadelphia, PA (US); Yuri Pekarsky, Philadelphia, PA (US)

Correspondence Address: THOMAS JEFFERSON UNIVERSITY, 1020 Walnut Street - Suite 630, Philadelphia, PA, 19107-5587, US

(21) Appl. No.: 09/855,294

(22) Filed: May 15, 2001

Related U.S. Application Data
(60) Provisional application No. 60/204,713, filed on May 16, 2000.

(51) Int. Cl. G01N 33/53; C12N 9/00; C07H 21/04; C12N 1/18; C12P 21/02; A61K 38/16; C07K 16/00; C07K 14/00

(52) U.S. Cl. 435/7.1; 435/183; 435/254.2; 435/69.1; 435/320.1; 536/23.2; 530/350; 514/12; 530/388.26

(57) ABSTRACT

In invertebrates, Fhit is encoded as a fusion protein with Nit. Outside of invertebrates, Nit homologs are found as separate polypeptides in organisms with Fhit homologs. Therefore, Nit and Fhit are expected to interact physically and function in the same cellular pathway. The structure of the NitFhit fusion protein and interactions between the Nit and Fhit polypeptides are defined. The present invention relates to the identification of small molecules that interact with, and regulate, the Nit protein. The present invention further relates to therapeutic compositions and their uses in regulating Nit activity, thereby modulating cellular proliferation.

### CROSS REFERENCE TO RELATED APPLICATIONS

This application claims priority under 35 U.S.C. 119 based upon U.S. Provisional Application No. 60/204,713, filed May 16, 2000.

### GOVERNMENT RIGHTS IN THE INVENTION

This invention was made in part with government support under Grant number CA75954 awarded by the National Institutes of Health.

### FIELD OF THE INVENTION

The present invention generally relates to the fields of molecular and structural biology and, more particularly, to the interaction between Nit and Fhit proteins and the subsequent regulation of cell proliferation.

### BACKGROUND OF THE INVENTION

Loss of Fhit protein is a frequent and early event in the development of lung cancer, the leading cause of cancer deaths worldwide (Sozzi, G., et al., Cell 85: 17-26, 1996; Mao, L., et al., J. Natl. Cancer Inst. 89: 857-862, 1997; Sozzi, G., et al., Cancer Research 58: 5032-5037, 1998; Huebner, K., et al., Advances in Oncology 15: 3-10, 1999). FHIT is located at 3p14.2 (Ohta, M., et al., Cell 84: 587-597 1996) and spans FRA3B (Zimonjic, D. B., et al., Cancer Research 57: 1166-1170, 1997) the most fragile site in the human genome. The instability of the giant FHIT locus, spanning more than 1.5 Mb of DNA (Mimori, K., et al., Proc. Natl. Acad. Sci. USA 96: 7456-7461, 1999) coupled with the small size of the transcript and coding region (Ohta, M., et al., Cell 84: 587-597, 1996) account for inactivation of the gene principally by deletion (Mimori, K., et al., Proc. Natl. Acad. Sci. USA 96: 7456-7461, 1999) and, less frequently, by methylation (Tanaka, H., et al., Cancer Research 58: 3429-3434, 1998) rather than by point mutation. Mutations in the first FHIT allele are either inherited as a t(3;8) translocation (Ohta, M., et al., Cell 84: 587-597, 1996; Huebner, K., et al., Ann. Rev. Genet. 32: 7-31, 1998) or are acquired by exposure to tobacco carcinogens (Sozzi, G., et al., Cancer Research 58: 5032-5037, 1998) papilloma virus insertion (Greenspan, D. L., et al., Cancer Research 57: 4692-4698, 1997) or other mechanisms. In the family carrying a t(3;8) translocation, affected young adults suffer bilateral, multifocal renal carcinomas (Cohen, A. J., et al., N. Engl. J. Med. 301: 592-595, 1979). Somatic loss of Fhit in humans is associated with cancers in a wide variety of sites including lung, kidney (Xiao, G. H., et al., Am. J. Pathol. 151: 1541-1547, 1997), stomach (Baffa, R., et al., Cancer Research 58: 4708-4714, 1998), pancreas (Simon, B., et al., Cancer Research. 58: 1583-1587, 1998), cervix (Greenspan, D. L., et al., Cancer Research 57: 4692-4698, 1997), ovary (Mandai, M., et al., Eur. J. Cancer 34: 745-749, 1998), head and neck (Virgilio, L., et al., Proc. Natl. Acad. Sci. USA 93: 9770-9775, 1996), breast (Ingvarsson, S., et al., Cancer Research 59: 2682-2689, 1999; Campiglio, M., et al., Cancer Research 59: 3866-3869, 1999) and hematopoetic cells. (Iwai, T., et al., Cancer Research 58: 5182-5187, 1998; Peters, U. R., et al., Oncogene 18: 79-85, 1999; Hallas, C., et al., Clin. Cancer Res. 5: 2409-2414, 1999). In addition, loss of Fhit is found in murine cancer cell lines (Pelkarsky, Y., et al., Cancer Research 58: 3401-3408, 1998) and targeted disruption of murine Fhit predisposes to stomach and sebaceous tumors in a pattern that resembles human Muir-Torre syndrome. (Fong, L. Y. Y., et al., Proc Natl Acad Sci USA 97: 4742-4747 2000).

Fhit is a member of the histidine triad (HIT) superfamily of nucleotide-binding proteins. (Brenner, C., et al., Nat. Struc. Biol. 4: 231-238, 1997; Brenner, C., et al., J. Cell Physiol. 181, 179-187 1999). Members of the Fhit branch of the HIT superfamily bind and cleave diadenosine polyphosphates (ApₙA) such as AppppA and ApppA to generate AMP plus ATP and ADP, respectively (Huang, Y., et al., Biochem. J. 312: 925-932, 1995; Barnes, L. D., et al., Biochem. 35: 11529-11535, 1996). The tumor-suppressing function of Fhit does not depend on cleavage of ApₙA (Siprashvili, Z., et al., Proc. Natl. Acad. Sci. USA 94: 13771-13776, 1997). The H96N allele of Fhit, which maintains micromolar binding to ApppA at the expense of a million-fold loss in catalytic activity (Pace, H. C., et al., Proc. Natl. Acad. Sci. USA 95: 5484-5489, 1998), is functional in tumor suppression (Siprashvili, Z., et al., Proc. Natl. Acad. Sci. USA 94: 13771-13776, 1997; Werner, N. S., et al., Cancer Research 60: 2780-2785, 2000). Fhit binds two ApₙA substrates per dimer, presenting all of the phosphates and two adenosines on a surface of the protein that is spatially and electrostatically altered in the substrate-bound form (Pace, H. C., et al., Proc. Natl. Acad. Sci. USA 95: 5484-5489, 1998). Thus, by analogy with G-proteins, Fhit has been proposed to function as a nucleotide substrate-dependent molecular switch (Brenner, C., et al., Nat. Struc. Biol. 4: 231-238, 1997; Pace, H. C., et al., Proc. Natl. Acad. Sci. USA 95: 5484-5489, 1998).

Re-expression of Fhit in cancer cell lines with FHIT deletions induces apoptosis (Ji, L., et al., Cancer Research 59: 3333-3339, 1999; Sard, L., et al., Proc. Natl. Acad. Sci. USA 96: 8489-8492, 1999), via an unknown mechanism. Thus, identification of molecules that interact with Fhit and/or participate in Fhit-dependent pathways is of great interest. Recently, a general method was proposed to identify interacting proteins by identifying a “Rosetta Stone” protein consisting of two unrelated proteins fused in one organism but expressed as separate polypeptides in other organisms (Marcotte, E. M., et al., Science 285:751-753, 1999). With few exceptions, experimental evidence and bioinformatic inference suggest that the existence of a fusion protein in one genome powerfully predicts that the separate polypeptides function in the same cellular or biochemical pathway in other organisms (Marcotte, E. M., et al., Science 285: 751-753, 1999; Enright, A., et al., Nature 402: 86-90, 1999). The strongest case that Rosetta Stone proteins decode real interactions can be made when the separate genes have similar gene expression patterns (Marcotte, E. M., et al., Science 285: 751-753, 1999) and are found in the same subset of genomes (i.e., share a phylogenetic profile) (Pellegrini, M., et al., Proc. Natl. Acad. Sci. USA 96: 4285-4288, 1999).

In mammals (Ohta, M., et al., Cell 84: 587-597, 1996; Pekarsky, Y., et al., Cancer Research 58: 3401-3408,1998) and fungi (Brenner, C., et al., Nat. Struc. Biol. 4: 231-238, 1997; Huang, Y., et al., Biochem J 312: 925-932, 1995). Fhit homologs are encoded as single polypeptides that are at least 42% identical within a core of 113 residues. In flies and worms, Fhit homologous domains are encoded at the C-termini of 460 and 440 amino acid polypeptides in which the 300 amino acid N-terminal domains are 22% identical to plant and bacterial nitrilases (Pekarsky, Y., et al., Proc. Natl. Acad. Sci. USA 95: 8744-8749, 1998), enzymes that hydrolyze compounds such as indoleacetonitrile to indoleacetic acid plus ammonia (Normanly, J., et al., Plant Cell 9: 1781-1790, 1997). Using Nit domains of fly and worm NitFhit as search molecules, murine and human orthologs are identified and found to be encoded as separate polypeptides that are 48% identical to invertebrate Nit domains (Pekarsky, Y., et al., Proc. Natl. Acad. Sci. USA 95: 8744-8749, 1998). This branch of the nitrilase superfamily is refered to as Nit proteins.

Satisfying the first criterion for the likely functional significance of natural fusion proteins (Marcotte, E. M., et al., Science 285: 751-753, 1999), the levels of Nit1 and Fhit mRNA are highly correlated in seven of eight tissues examined in mouse, the exception being brain, which has a high level of Fhit and a low level of Nit1 message (Pekarsky, Y., et al., Proc. Natl. Acad. Sci. USA 95: 8744-8749, 1998). To address the second criterion (Marcotte, E. M., et al., Science 285: 751-753, 1999), an additional Nit protein was cloned from human (SEQ. ID NO: 1) and mouse (SEQ. ID NO: 2), a Nit homolog from frog (SEQ. ID NO: 3), two homologs, Nit2 and Nit3, from budding yeast (SEQ. ID NO: 4 and SEQ. ID NO: 5, respectively), and identify two homologs, Nit1 and Nit2, from fission yeast (SEQ. ID NO: 6 and SEQ. ID NO: 7, respectively). Thus, Nit homologs, having been identified in vertebrate and invertebrate animals and fungi (FIG. 1), cover the same phylogenetic space as Fhit homologs (Brenner, C., et al., Nat. Struc. Biol. 4: 231-238, 1997; Huang, Y., et al., Biochem J 312: 925-932, 1995).

Further, worm NitFhit was purified by following the GpppBODIPY-hydrolysis activity (Draganescu, A., et al., J Biol Chem 275: 4555-4560, 2000) of its Fhit active site. The nucleotide-specificity of the Fhit active site was characterize, illustrating that NitFhit prefers AppppA>ApppA>ApppppA>pyrophosphate>other compounds. Finally, the crystal structure of NitFhit was determined, defining a new α-β-β-α sandwich protein fold for the nitrilase superfamily. Nit possesses a novel tetrameric superstructure, termed a beta box, that recognizes a pair of Fhit dimers at opposite poles. Nit and Fhit domains are not merely tethered together in the fusion protein. In addition, the most C-terminal β-strand encoded by the Nit portion of the NitFhit sequence extends out of the Nit globular domain and binds Fhit.

### SUMMARY OF THE INVENTION

It is an object of the present invention to present an isolated nucleic acid encoding a human Nit2 protein, the nucleotide sequence is being a cDNA sequence. In one embodiment the isolated nucleic acid sequence encodes a human Nit2 protein having an amino acid sequence of SEQ. ID. NO: 1. It is a further object of the invention that the isolated nucleic acid encoding the human Nit2 protein has the nucleotide sequence of SEQ. ID. NO: 8.

It is a further object of the present invention to provide a human Nit2 protein. In one embodiment the isolated protein has the amino acid sequence of SEQ. ID. NO: 1.

It is another object of the present invention to provide an antibody which specifically binds to an epitope of a human Nit2 protein.

It is an object of the present invention to present an isolated nucleic acid encoding a mouse Nit2 protein, the nucleotide sequence is being a cDNA sequence. In one embodiment the isolated nucleic acid sequence encodes a mouse Nit2 protein having an amino acid sequence of SEQ. ID. NO: 2. It is a further object of the invention that the isolated nucleic acid encoding the mouse Nit2 protein has the nucleotide sequence of SEQ. ID. NO:9.

It is a further object of the present invention to provide a mouse Nit2 protein. In one embodiment the isolated protein has the amino acid sequence of SEQ. ID. NO: 2.

It is another object of the present invention to provide an antibody which specifically binds to an epitope of a mouse Nit2 protein.

It is an object of the present invention to provide an S. pombe Nit2 protein. In one embodiment the isolated S. pombe Nit2 protein has the amino acid sequence of SEQ. ID. NO: 7. It is a further object of the present invention to provide an antibody which specifically binds to an epitope of the S. pombe Nit2 protein.

It is an object of the present invention to provide an S. cerevisiae Nit3 protein. In one embodiment the isolated S. cerevisiae Nit3 protein has the amino acid sequence of SEQ. ID. NO: 5. It is a further object of the present invention to provide an antibody which specifically binds to an epitope of the S. cerevisiae Nit3 protein.

It is an object of the present invention to present an isolated nucleic acid encoding a X. laevis Nit1 protein, the nucleotide sequence being a cDNA sequence. In one embodiment the isolated nucleic acid sequence encodes a X. laevis Nit1 protein having an amino acid sequence of SEQ. ID. NO: 3. It is a further object of the invention that the isolated nucleic acid encoding the X. laevis Nit1 protein has the nucleotide sequence of SEQ. ID. NO: 10.

It is a further object of the present invention to provide a X. laevis Nit1 protein. In one embodiment the isolated protein has the amino acid sequence of SEQ. ID. NO: 3.

It is another object of the present invention to provide an antibody which specifically binds to an epitope of a X. laevis Nit1 protein.

It is an object of the present invention to provide an S. pombe Nit1 protein. In one embodiment the isolated S. pombe Nit1 protein has the amino acid sequence of SEQ. ID. NO: 6. It is a further object of the present invention to provide an antibody which specifically binds to an epitope of the S. pombe Nit1 protein.

It is an object of the present invention to provide an S. cerevisiae Nit2 protein. In one embodiment the isolated S. cerevisiae Nit2 protein has the amino acid sequence of SEQ. ID. NO: 4. It is a further object of the present invention to provide an antibody which specifically binds to an epitope of the S. cerevisiae Nit2 protein.

It is an object of the present invention to provide a method of identifying a molecule that specifically binds to a Nit2 protein and is functionally active in mimicking a Fhit interact. The Nit2 is brought into contact with a plurality of molecules under conditions conducive to binding between the Nit2 and the molecules. Molecule(s) that specifically bind to the Nit2, and are functionally active in mimicking the Fhit interaction, are thereby identified. In one embodiment a Fhit mimic binds to a Nit2 protein in any cell and is functionally active in mimicking a Fhit interaction.

It is an object of the present invention to provide a method of treating a disease state in which an activity of a Nit2 protein is altered in a mammal. Administration of a therapeutically effective amount of a Fhit mimic, where the Fhit mimic binds to the Nit2 protein, will induce programmed cell death. In one embodiment of the present invention the disease is a proliferative disorder.

It is another object of the present invention to provide a pharmaceutical composition of a Fhit mimic.

It is an object of the present invention to provide a method of identifying a molecule that specifically binds to a Nit2 protein and is functionally active in antagonizing a Fhit interaction. The Nit2 is brought into contact with a plurality of molecules under conditions conducive to binding between the Nit2 and the molecules. A molecule within the plurality of molecules that specifically binds to the Nit2, and is functionally active in antagonizing the Fhit interaction, is thereby identified.

It is an object of the present invention to provide a Fhit antagonist that binds to a Nit2 protein in any cell and is functionally active in antagonizing a Fhit interaction.

It is an object of the present invention to provide a method of treating a disease state in which an activity of a Nit2 protein is altered in a mammal. A therapeutically effective amount of a Fhit antagonist is administered to the mammal and binds to the Nit2 protein, thereby promoting cell proliferation. In one embodiment the disease is a degenerative disease.

The present invention also provides a pharmaceutical composition of a Fhit antagonist.

### DESCRIPTION OF THE DRAWINGS

FIG. 1. Sequence Alignment of Nit Proteins with a Plant Nitrilase.

The Nit domains of C. elegans and D. melanogaster NitFhit proteins are aligned with Nit homologs from H. sapiens, M. musculus, S. pombe, S. cerevisiae, X. laevis, and Nitrilase1 from A. thaliana. Secondary structural elements and sequence numbers correspond to worm NitFhit. The figure was prepared with ALSCRIPT (Levitt, M., Chothia, C., Nature 261: 552-558, 1976). Human Nit2, murine Nit2, frog Nit1, and budding yeast Nit2 and Nit3 are newly cloned and have been deposited in Genbank with accession numbers VVV, WWW, XXX, YYY and ZZZ. Carets mark the positions of insertions that are found in some of the sequences. Residues found in the vicinity of Cys 169 are indicated by filled circles.

FIG. 2. Structure Determination of NitFhit.

a, A portion of the 2.8 Å experimental electron density map in stereo. The map was contoured at 1.5 σ and superimposed on the refined atomic model.

b, Stereo ribbon view of the Nit domain of a NitFhit monomer (northern conformation). Secondary structural elements are indicated. The Fhit domain, C-terminal to the Nit domain, and three additional subunits of the NitFhit tetramer are not shown.

FIG. 3. Structure of the NitFhit tetramer.

a, A Levitt and Chothia (Levitt, M., Chothia, C. Nature 261: 552-558, 1976) representation of the NitFhit tetramer. The 222 point symmetry of the tetramer is indicated by the manner in which labels are flipped across symmetry axes.

b, Stereo representation of the NitFhit tetramer. NitFhit monomers are colored green and blue in the northern hemisphere, red and yellow in the southern hemisphere. Nit domains (residues 10 to 296) are in bold colors and Fhit domains (residues 297 to 440) are in pastel colors. The boldly colored elements in the northern and southern domains are portions of Nit that interact with Fhit.

FIG. 4. Nit Fits.

Molecular features of the physical interaction between Nit and Fhit. At the north and south poles of the Nit tetramer, pairs of antiparallel NS 13 Nit strands (bold colors) interact with Fhit domains (pastel colors) beneath pairs of antiparallel FH1 helices.

FIG. 5. Structural Plasticity in Nit Tetramers

Two conformations of the second Nit helix, NH2. Portions of two non-identical Nit domains were superimposed to show two conformations at a solvent-exposed surface of the NitFhit tetramer. Without mercury binding, the segment is not helical. With mercury bound, the helix is bent. Both conformations distort helix NH2 at a site in which other Nit sequences, but not worm NitFhit, contain Gly and/or Pro residues.

FIG. 6. Putative Nit Active Site

The region around Cys 169, a residue conserved in nitrilases, has a distinct pattern of conservation in Nit proteins. Residues aligning with Cys 169, Glu 54 and Lys 127 are predicted to form a catalytic triad with Glu as the general base.

### DESCRIPTION OF THE INVENTION

Methods

Protein Expression and Purification.

The C. elegans NitFhit cDNA was amplified with primers that generated an NdeI site at the initiator codon and an XhoI site 3′ of the stop codon. After restriction with NdeI and XhoI, the fragment was ligated to plasmid pSGA02 (Ghosh, S., Lowenstein, J. M. Gene 176: 249-255, 1997) digested with the same enzymes.

Protein was expressed in E. coli strain ER2566 (New England Biolabs) grown in LB with 150 μg ml⁻¹ ampicillin. 1 I cultures, shaken at 24° C. in 2800 ml Fernbach flasks, were induced with 0.4 mM IPTG at an optical density (λ=600 nm) of 0.4 and aerated for nine hours. Steps before and including ammonium sulfate precipitation were performed at 0 to 4° C. Frozen cell pellets were resuspended in 50 mM NaHEPES, pH 7.0, 5 mM DTT, 10% glycerol, 0.5 mM PMSF, 2 μg ml⁻¹ leupeptin and 3.4 μg ml⁻¹ pepstatin, and lysed by sonication. Cleared lysate was subjected to protamine sulfate precipitation followed by centrifugation to remove nucleic acids. A 20-60% ammonium sulfate fraction was obtained, and resuspended and dialyzed into 50 mM NaHEPES pH 7.2, 5 mM DTT, 10% glycerol. The dialysate was loaded onto a 58 ml POROS 20 HQ column (PE Biosystems) with 50 mM NaHEPES, pH 7.2, 5 mM DTT, 2% glycerol as running buffer and NaCl as eluant.

GpppBODIPY hydrolysis activity eluted at 0.2 M NaCl. Peak fractions were pooled and buffer was exchanged to 25 mM NaHEPES pH 7.0, 5 mM DTT, 2% glycerol using 10,000 Da-retaining Ultrafree filters (Millipore). Concentrated and desalted sample was loaded onto a 1.7 ml POROS 20 CM column (PE Biosystems) and chromatographed as before, with the peak of total protein eluting with enzymatic activity at 0.25 M NaCl.

Purified NitFhit was concentrated to 7 mg ml⁻¹ in 10 mM NaHEPES, pH 7.0, 50 mM NaCl, 5 mM DTT, microaliquoted, and stored at −80° C. Data from Edman degradation, mass spectrometry and analytical ultracentrifugation indicated that purified NitFhit contains an intact N-terminal Met, displays mass/charge ratios consistent with the predicted monomer size of 49,936 Da, and exists as a stable 200,000 Da tetramer in solution independent of nucleotide occupancy.

Characterization of the Fhit Active Site of NitFhit.

Standard activity assays of the Fhit active site of NitFhit used GpppBODIPY in an initial rate assay as developed for Fhit (Draganescu, A. J. Biol Chem. 275: 4555-4560, 2000) except that reactions were initiated by addition of 10-50 ng of total protein diluted into 20 mM NaHEPES pH 7.0, 10% glycerol, 5 mM DTT, 0.2 mg ml ml⁻¹ BSA and incubated at 21° C. k_{cat}/K_{M} determination with ApppBODIPY was performed as a substrate decay assay (Draganescu, A. J Biol Chem 275: 4555-4560, 2000) with 1.5 μM ApppBODIPY, initiated by 0.15 μmol NitFhit. For GpppBODIPY, substrate concentration was titrated from 40 μM to 2.5 μM, and k_{cat} and K_{M} were determined from initial rates using 0.5 μmol of enzyme (Draganescu, A. J. Biol. Chem. 275: 4555-4560, 2000). Assays of nonlabeled compounds as competitive inhibitors of ApppBODIPY hydrolysis were also performed as developed for human Fhit. KM values for ApppA, AppppA, ApppppA, ATP-αS, GTP-αS and K₁ values for pyrophosphate, monophosphate and AMP were derived from titration of each nonlabeled compound into 1.5 μM ApppBODIPY assays at five concentrations of the competitors (Draganescu, A. J Biol Chem 275: 4555-4560, 2000).

Protein X-Ray Crystallography.

NitFhit crystals were grown by hanging drop vapor diffusion by mixing 2 μl of 7 mg ml⁻¹ protein in 10 mM NaHEPES, pH 7.0, 50 mM NaCl, 5 mM DTT with 2 μl of 38% 2-methyl-2,4-pentanediol (MPD) and equilibrating against 1 ml 38% MPD. After one week at room temperature, individual microcrystals were seeded into similar drops, equilibrated against 35% MPD, and grown for one month. Crystals (˜100 μm×200 μm×300 μm) were flash-frozen in liquid nitrogen.

Based on data collected at Cornell High Energy Synchrotron Source beamline F-1, native crystals had the symmetry of space group 1222 or I2₁2₁2₁, contained a NitFhit monomer in the asymmetric unit and 58% solvent, and were ordered to 3.5 Å resolution. A single frozen crystal was thawed into 10 μl 5 mM NaBEPES, 25 mM NaCl, 2.5 mM DTT, 20% MPD with 1 mM thimerosal, and refrozen after 8 hrs. At the National Synchrotron Light Source, beam X8-C was tuned to the measured absorbtion edge for the mercurated crystal (λ_{edge}=1.008989 Å) and to a remote wavelength (λᵣₑₘₒₜₑ=0.992782 Å), and X-ray diffraction data were measured in 1° oscillations with an ADSC Quantum-4 CCD camera 250 mm from the crystal. Data, indexed and scaled with the HKL package (Otwinowski, Z., Minor,W., Gene 176: 249-255, 1997), indicated the presence of mercury atoms, a reduction in crystallographic symmetry to P2₁2₁2 (from I222 The change in space group was accompanied by a 6% reduction in the b cell length a 2% reduction in solvent content, and an increase in resolution to 2.8 Å.

Using the CNS package (Brunger, A. T., et al., Acta Crystallogr. D Biol. Crystallogr. 54: 905-921, 1998) a four atom mercury solution was obtained for the λ_{edge} anomalous difference Patterson map. Using diffraction data from both wavelengths, the heavy atom positions and the scattering factors f′ and f″ of the mercurated protein were refined (Brunger, A. T., et al., Acta Crystallogr. D Biol. Crystallogr. 54: 905-921, 1998) and used to generate TAD phases (Table 1). The density-modified TAD electron-density map of the refined, enantiomorphic heavy atom solution was interpretable. A fifth mercury position, located in this map, was used to generate the final density-modified TAD electron density map (hereafter, the experimental map) that was used for model building (FIG. 3a) with O, a software package used for model building (http://www.imsb.au.dk/˜mok/o/). Electron density corresponding to two nonidentical Fhit dimers was located. Each Fhit dimer occurs across a crystallographic two-fold rotation axis such that the east and west subunits of Fhit are identical. The two non-identical Fhit dimers occur as an imperfect 222 tetramer with an origin of noncrystallographic symmetry (NCS) at 0.5, 0.5, 0.25.

TABLE 1
X-Ray Data Collection, Phasing and Refinement Statistics
Data Collection and Phasing of P2,2,2-thimerosol crystals

| | λ_{edge} | | λᵣₑₘₒₜₑ | |
|---|---|---|---|---|
| Resolution, Å | 30.0-2.8 | | 30.0-2.8 | |
| Completeness, % (outer s | 97.9 (91.4) | | 97.6 (90.3) | |
| Multiplicity | 7.5 | | 7.0 | |
| I/σ | 24.6 | | 23.2 | |
| R_{sym}, % (outer shell) | 4.5 (8.8) | | 5.1 (9.9) | |
| Rₐₙₒₘ, % (outer shell) | 3.8 (6.2) | | 5.0 (7.5) | |
| Hg sites; Phasing Power; | 5; | 1.95; | 0.47; | 0.96 |
| FOM; FOMDM | | | | |

| | |
|---|---|
| Refinement Statistics | |
| Nonhydrogen atoms (water | 6708 (161) |
| molecules) | |
| Unique reflections (free) | 49,386 (3315) |
| R_{work}, % (R_{free}) | 19.0 (23.1) |
| msd bond lengths, Å R | 0.007 |
| Rmsd bond angles, ° | 1.4 |
| Average B value, Å² | 28.5 |

The experimental map was of sufficient quality to build from residues 13 to the C-terminus of each nonidentical, 440 amino acid polypeptide with 23 residues missing from the Fhit domain of one molecule and 30 residues missing from the other. Nonidentical NitFhit molecules were built independently and refined without NCS restraints. The protein atomic model, containing additionally five mercury atoms and a bulk solvent model, was refined by simulated annealing (Brunger, A. T., et al., Acta Crystallogr. D Biol. Crystallogr. 54: 905-921, 1998) against λ_{edge} data with a maximal-likelihood target function based on experimental phases (Pannu, N. S., et al., Acta Crystallogr. D Biol. Crystallogr. 54: 1285-1294, 1998). All reflections from 30.0 Å to 2.8 Å were included in refinement except 7% reserved for free R factor analysis⁵⁶. The five mercury atoms appear to be ethylmercuric adducts to cysteine residues 55, 75 and 169 of the (northern) A chain and residues 55 and 169 of the (southern) B chain. These adducts were built and refined as ethylmercury with occupancies of 40% to 57%. Nineteen additional amino acids, 161 water molecules, and four sodium ions, and one ordered MPD molecule were built from sigma A weighted, cross-validated, phase combined, 2Fo-Fc and Fo-Fc maps. (Brunger, A. T., et al., Acta Crystallogr. D Biol. Crystallogr. 54: 905-921, 1998). The final atomic model has an R_{work} of 19.0% and an R_{free} of 23.1% with geometry that is neither under-restrained nor over-restrained with respect to ten recently released protein structures refined to 2.8 Å. The current model has an R_{work} of 20.4% and an R_{fee} of 24.5% with geometry that is neither under-restrained nor over-restrained with respect to ten recently released protein structures refined to 2.8 Å. Molecular graphics methods were as described (Pace, H. C., et al., Proc. Natl. Acad. Sci. USA 95: 5485-5489, 1998).

Coordinates.

The coordinates (1EMS) and structure factors (1EMSsf) have been deposited into the Protein Data Bank.

Results

Nit Homologs are Found in the Same Organisms as Fhit Homologs

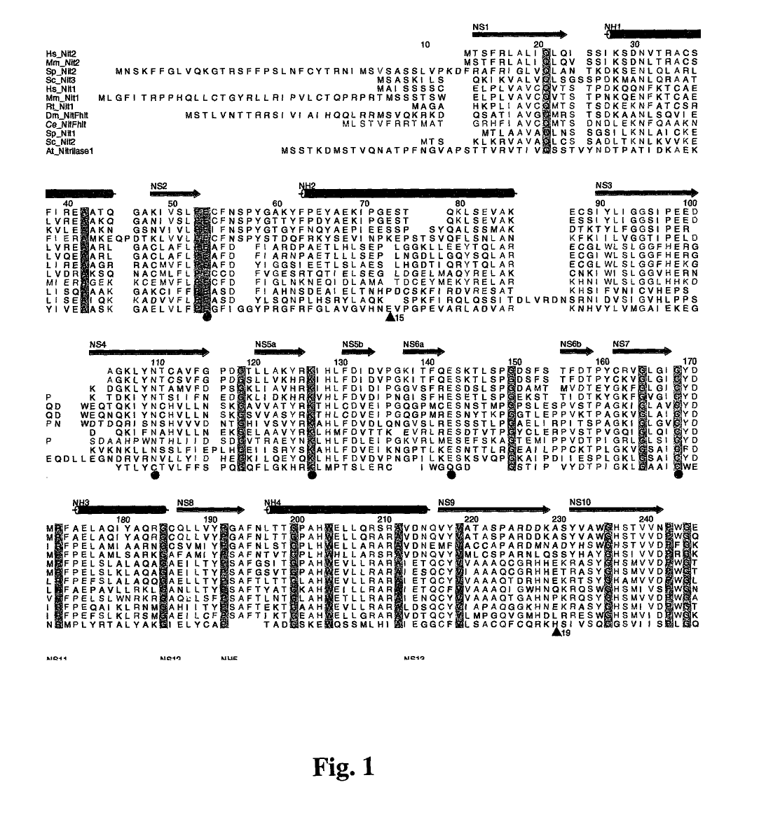

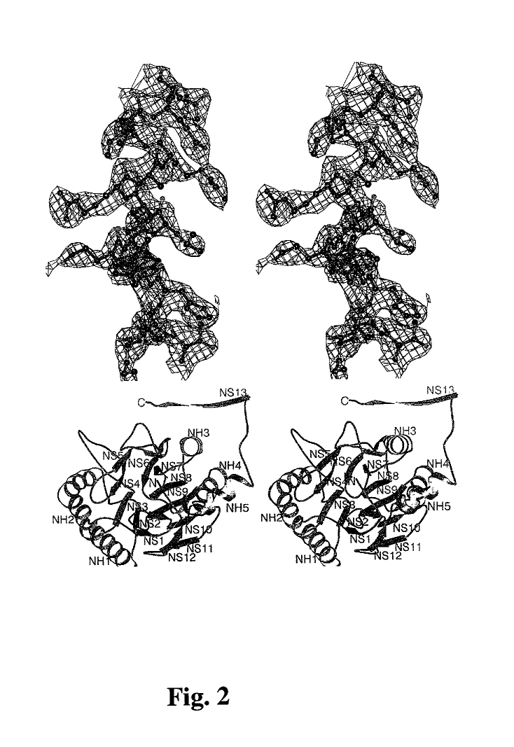

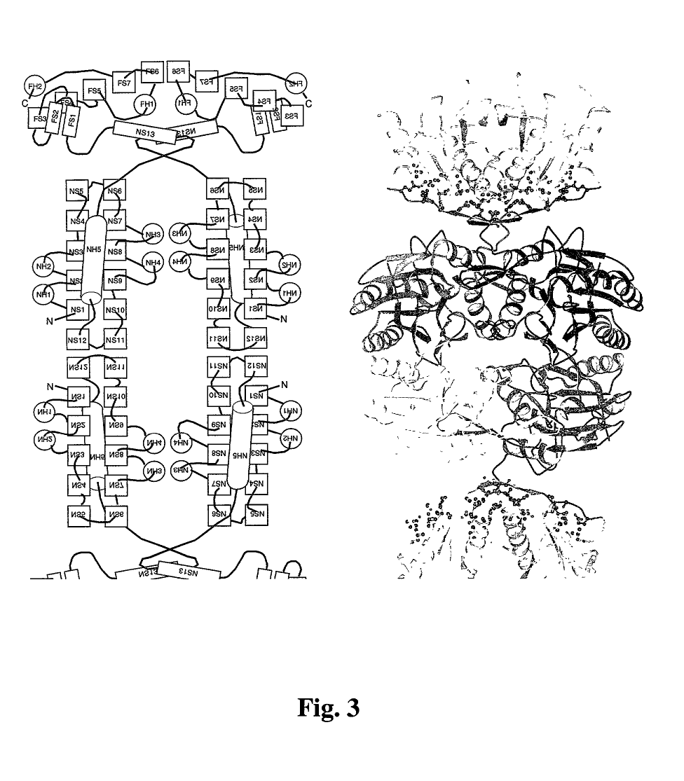

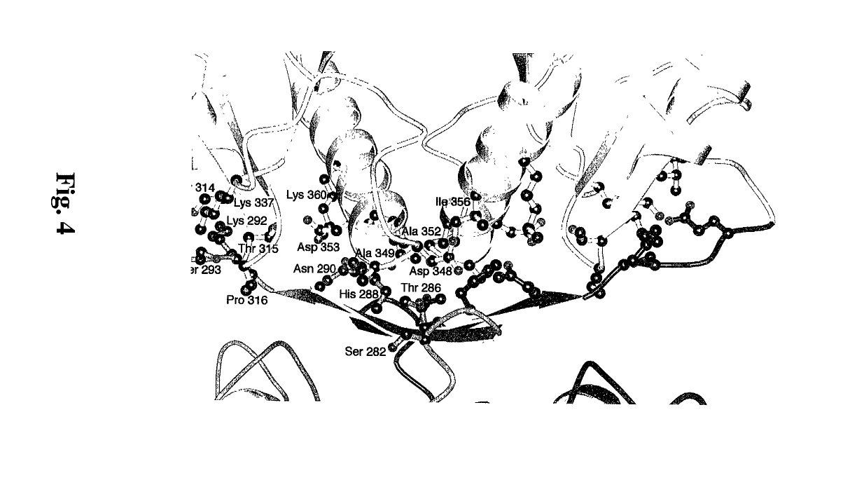

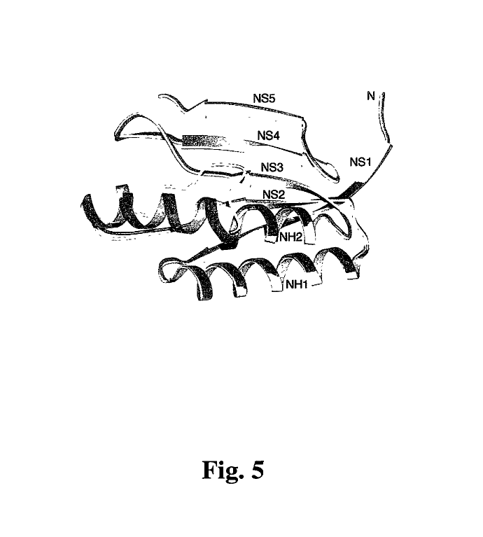

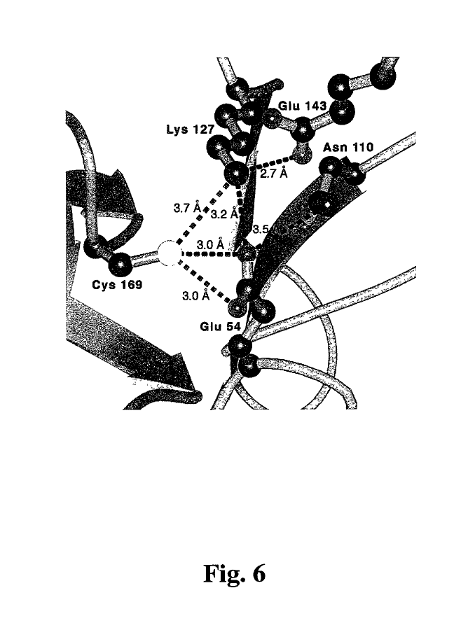

In the course of cloning Fhit homologous cDNAs from D. melanogaster and C. elegans, NitFhit sequences were identified (Pekarsky, Y. et al., Proc. Natl. Acad. Sci. 95: 8744-8749, 1998). The Nit domain of the invertebrate NitFhit proteins was classified as a distinct member of the nitrilase superfamily and used to clone the single most homologous sequences from human and mouse cDNA libraries (Pekarsky, Y., et al., Proc. Natl. Acad. Sci 95: 8744-8749, 1998). It has been pointed out that events that fuse unrelated proteins (Marcotte, E. M., et al., Science 285: 751-753, 1999; Enright, A., et al., Nature 402: 86-90, 1999) are most likely to be functionally significant (Marcotte, E., et al., Nature 402: 83-86, 1999) if the separate proteins have similar gene expression patterns and have similar phylogenetic profiles (Pellegrini, M., et al., Proc. Natl. Acad. Sci. USA 96: 4285-4288, 1999). At the level of tissue-specificity, murine Fhit and Nit1 have nearly identical mRNA accumulation profiles (Pekarsky, Y., et al, Proc. Natl. Acad. Sci. USA 95: 8744-8749, 1998). Therefore, identification of Nit-related genes from divergent organisms known to contain Fhit-homologous genes, namely S. cerevisiae and S. pombe (Brenner, C., et al., Natl. Struc. Biol. 4: 321-238, 1997) and X. laevis, was sought. In each yeast, two Nit-related sequences were identified (FIG. 1), as well as sequences related to plant nitrilases. The frog also yielded a Nit sequence and further examination of human and murine expressed sequence tag databases allowed for the identification of a second Nit coding sequence from human and mouse (FIG. 1). Nit sequences have a low level of identity with nitrilases and a substantial level of identity with each other. Nit homologs, having been found fused or coordinately expressed with Fhit homologs (Pekarsky, Y., et al., Cancer Research 58: 3401-3408, 1998) and in the same organisms as Fhit homologs (FIG. 1), are reasonable candidates for proteins that interact with Fhit homologs.

Characterization of the Fhit Active Site of NitFhit

When D. melanogaster NitFhit is expressed in E. coli (Pekarsky, Y., et al., Cancer Research 58: 3401-3408, 1998) it is insoluble, therefore worm NitFhit, the product of the nft-I gene of C. elegans (Pekarsky, Y., et al., Cancer Research 58: 3401-3408, 1998) was expressed. The enzyme was followed fluorimetrically with Gppp-S-(4-4-fluoro-5,7-dimethyl-4-bora-3a,4a-diaza-s-indacine-3-yl) GpppBODIPY) (Draganescu, A., et al., J Biol Chem 275, 4555-4560, 2000), a quenched fluorescent nucleotide substrate developed for use with Fhit. Worm NitFhit is ˜20% soluble when expressed in E. coli at 20° C. The conventional purification of NitFhit, based on maximizing GpppBODIPY-hydrolase specific activity, was performed exclusively from the soluble fraction (Table 2).

TABLE 2
Purification and Characterization of the Fhit Active Site of NitFhit
Purification of NitFhit via the Fhit Active Site

| | | Specific | Yield | |
|---|---|---|---|---|
| Total | | Activity | cum- | Purification |
| Protein | Units | pmol min⁻¹ | ulative | Cumulative |

| | | | | | |
|---|---|---|---|---|---|
| Fraction | mg | pmol min⁻¹ | mg⁻¹ | % | fold |

| | | | | | |
|---|---|---|---|---|---|
| Cleared | 764 | 8.00 E7 | 1.05 E5 | 100 | — |
| lysate | | | | | |
| Am- | 451 | 5.60 E7 | 1.24 E5 | 70 | 1.2 |
| monium | | | | | |
| Sulfate | | | | | |
| HQ | 13.5 | 1.61 E7 | 1.19 E6 | 20 | 11.3 |
| CM | 8.9 | 2.12 E7 | 2.38 E6 | 27 | 22.7 |

Nucleotide Specificity of the Fhit Active Site of NitFhit

| | k_{cat}/K_{M}k_{cat} | K_{M} | K_{I} | |
|---|---|---|---|---|
| | (10⁵ s⁻¹ M⁻¹ | s⁻¹ | μM | μM |
| ApppBODIPY | 5.0 ± 0.1 | | | |
| GpppBODIPY | 6.3 | 2.4 | 3.7 | |
| ApppA | | | | 4.2 ± 0.4 |
| AppppA | | | | 2.7 ± 0.3 |
| ApppppA | | | | 4.5 ± 0.7 |
| pyrophosphate | | | | 78 ± 12 |
| AMP | | | | 153 ± 13 |
| ATP-αS | | | | 287 ± 73 |
| GTP-αS | | | | 481 ± 27 |
| monophosphate | | | | 2660 ± 490 |

The function of Fhit is thought to depend on formation of substrate complexes with ApₙA (Pace, H. C., et al., Proc. Natl. Acad. Sci. USA 95: 5484-5489, 1998) in the presence of higher cellular concentrations of purine mononucleotides and other competitors (Draganescu, A., et al., J Biol Chem 275: 4555-4560, 2000). To assess the nucleotide-specificity of the worm Fhit homolog, a series of assays with Appp-S-(4-4-fluoro-5,7-dimethyl-4-bora-3a,4a-diaza-s-indacine-3-yl) (ApppBODIPY) and GpppBODIPY were performed. Titration of nonlabeled nucleotides and related compounds into fluorescent nucleotide-hydrolysis assays allows determination of the binding constant of each compound for the Fhit active site (Draganescu, A., et al., J Biol Chem 275: 4555-4560, 2000). As shown in Table 2, the nucleotide specificity of the Fhit domain of NitFhit is similar to that of Fhit. Whereas human Fhit has a slight binding preference for ApppA over AppppA, the worm enzyme, like the homolog from fission yeast (Robinson, A. K., et al., Biochemica et Biophysica Acta 1161: 139-148, 1993), prefers AppppA. As is the case for the human enzyme, pyrophosphate competes for the Fhit active site of NitFhit more effectively than do purine mononucleotides. After ApppA and AppppA, both enzymes prefer ApppppA>pyrophosphate>AMP and ATP-αS>GTP-αS>monophosphate. k_{cat}/Kₘ, the single most important measure of an enzyme's activity on a substrate, was measured for ApppBODIPY and GpppBODIPY in substrate decay assays and initial-rate assays, respectively (Draganescu, A., et al., J Biol Chem 275: 4555-4560, 2000). While worm NitFhit displays only 22% of the activity of human Fhit on ApppBODIPY, it displays 109% of the activity of human Fhit on GpppBODIPY. Thus, the Nit domain of NitFhit does not inhibit the nucleotide-binding or hydrolysis activity of the associated Fhit domain.

Nit is a Novel α-β-β-α Sandwich Protein

To determine the structure of Nit and the nature of Nit-Fhit interactions, worm NitFhit was crystallized and its crystal structure was determined. The 440 amino acid polypeptide (molecular weight=49,936 Da) has a molecular weight of 200,000 Da in solution (see Methods) and crystallized with a monomer in the asymmetric unit in space group 1222. The symmetry of these crystals suggested that NitFhit tetramers are located at the origin and center of the unit cell and that their oligomeric symmetry consists of three mutually-perpendicular two-fold rotation axes. These crystals were large and single but diffracted weakly to no better than 3.5 Å resolution at synchrotron sources. Crystals soaked with thimerosal or ethylmercuric phosphate exhibited a reduction in crystallographic symmetry to P2₁2₁2, a concomitant increase in the size of the asymmetric unit to two monomers, a 6% reduction in the length of one unit cell length, and a corresponding 2% reduction in solvent content. These derivatized crystals showed a striking increase in reflection intensities and resolution. Diffraction data from a single thimerosal-soaked crystal, collected at the mercury absorbtion edge and at one other wavelength, were used to solve the structure of mercurated NitFhit by two-wavelength anomalous diffraction (TAD) phasing to 2.8 Å resolution (Table 1). The two nonidentical NitFhit monomers were built from a density-modified TAD electron density map (FIG. 2a) and refined independently.

By sequence alignment, the Nit domain of NitFhit spans from residue 1 through 296 (see ref. Pekarsky, Y., et al., Proc. Natl. Acad. Sci. USA 95: 8744-8749, 1998 and FIG. 1) and the Fhit domain spans from residue 297 to 440 (Brenner, C., et al., Nat. Struct. Biol. 4: 231-238, 1997; Brenner, C., et al., J. Cell Physiol. 181: 179-187, 1999; Draganescu, A., et al., J Biol Chem 275, 4555-4560 2000).

The unit cell measured a=68.74 Å, b=100.44 Å, c=158.65 Å. λ_{edge} was 1.008989 and λᵣₑₘₒₜₑ was 0.992782. The f′ and f″ scattering factors, refined from reference values of −17.79 e- and 7.24 e- to −13.49 e- and 9.41 e- for edge and −10.86 e- and 9.99 e- to −10.86 e- and 11.18 e- for λᵣₑₘₒₜₑ. R_{sym}=Σ|−<I>|/Σ<I> in which I is a measured intensity and <I> is the average intensity from multiple measurements of symmetry-related reflections. Rₐₙₒₘ=Σ|<F+>−<F−>|Σ<F> in which <F+> and <F−> are the average structure factors of Friedel pairs. Phasing Power, Figure of Merit (FOM), and Figure of Merit after Density Modification (FOMDM) were as defined in CNS⁵⁴ for anomalous difference phasing.

The Nit domain, defined by continuous electron density from residue 10 to its C-terminus, is a novel protein fold (FIG. 2b) consisting of five (α-helices designated NH1 to NH5 and 13 β-strands designated NS1 to NS13. In CATH (Class, Architecture, Topology, Homologous superfamily) nomenclature (Pekarsky, Y., et al., Cancer Research. 58: 3401-3408, 1998), Nit can be assigned to the α-β class and the 4-layer sandwich architecture and is the first of its kind in topology and superfamily. The core of Nit is a highly regular α-β-β-α sandwich structure containing helices NH1 through NH4 and strands NS1 through NS12 (FIG. 2b and FIG. 3a). A cross section of the Nit core reveals a layer containing two (x-helices, followed by two layers of 6 β-sheets, followed by a layer of two α-helices. The most similar of the 12-stranded α-β-β-α sandwich folds is that of DNase I (Lahm, A., Stuck, D. J. Mol. Biol. 222: 645-667, 1991) and related nucleases. However, the α-β-β-α sandwich of DNase I is topologically distinct from that of Nit and is unlikely to be related. The pattern and direction of the first 8 elements of the Nit core (NS1, NH1, NS2, NH2, NS3, NS4, NS5, NS6 with N-termini of NS1, NS2, NS3, NS5 and C-termini of NH1, NH2, NS4 and NS6 facing the viewer in FIG. 2b) are repeated by the second 8 elements (NS7 through NS12) by an internal pseudo two-fold rotation axis. C-terminal to NS12, Nit contains helix NH5 and strand NS13 orthogonal to the core. NS 13, extended away from the globular Nit core, makes extensive interactions with the Fhit domain, as discussed below.

At residue 297, the NitFhit polypeptide aligns with residue 1 of human Fhit. Human Fhit structures are defined for residues 2 to 106 and 128 to their C-termini at residue 147 (Pace, H. C., et al., Proc. Natl. Acad. Sci. USA 95: 5484-5489, 1998; Lima, C. D., et al., Structure 5: 763-774, 1997). The Fhit domain of worm Fhit contains the seven β-strands, FS1 through FS7, and the two α-helices, FH1 and FH2, of Fhit and is nearly identical to human Fhit in all respects. Refined NitFhit models contain a 20-residue gap in the same location as the 21-residue gap within human Fhit models. The largest root mean square differences between superimposed human Fhit and worm Fhit domain are in the loop between FH1 and FS6. Even there, the Cα positions differ by 2 Å or less.

Nit Tetramers form a 52-stranded Beta Box

Nit monomer domains pack into a tetramer that contains two types of homotypic Nit-Nit interfaces that are herein described as north-south and east-west (FIG. 3). Heavy atom binding made the northern and southern hemispheres conformationally different but did not disturb their perfect east-west symmetry. Fhit dimers are located at the north and south poles of the Nit tetramer.

The east-west dimer interface of Nit, parallel to an already extensive Fhit dimer interface formed by FH1 and FS6, is formed by a 4-helix bundle (NH3 and NH4 with their symmetry mates) and an antiparallel β-interaction mediated by NS 13 (FIG. 3). Thus, the east-west Nit dimer turns a four-layered α-β-β-α sandwich into an eight-layered α-β-β-α-α-β-β-α sandwich. Helices NH1 and NH2 are solvent-exposed on the external layers of the sandwich. Furthermore, one edge of the β-sheets is enclosed by NH5 while the other edge of the β-sheets is exposed to solvent. According to amino acid conservation detected (FIG. 1), it's expected that all Nit proteins form α-β-β-α-α-β-β-α dimers.

The north-south Nit interface is formed by antiparallel, homotypic β-interactions involving strands NS11 and NS12 (FIG. 3). These interactions double the width of the four β-sheets in theα-β-β-α-α-β-β-α sandwich from 6 strands north to south to 12 strands north to south. The tetrameric Nit assembly can be termed a 52-stranded beta box. The east and west sides of the beta box each consist of two 12-stranded β-sheets. Between the east and west sides are two 4-helix bundles. The north and south poles of the beta box are capped by the final β-strand (NS13) of each monomer as an anti-parallel pair of strands on each pole. By alignment, some of the salt bridges that stabilize the north-south dimer interface appear to be absent in homologous Nit sequences. Thus, it remains to be seen whether vertebrate and fungal Nit proteins will be dimers or tetramers.

Nit Sequences Bind Fhit

The C-terminal β-strand encoded by Nit sequences, NS13, exits the Nit core domain (FIG. 2b). Formation of the east-west Nit dimer allows NS13 strands to pair in an antiparallel fashion and formation of the north-south Nit tetramer allows these strands to form the top and bottom sides of the beta box (FIG. 3). Moreover, in worm NitFhit, the NS 13 elements have extensive interactions with Fhit dimers and appear to be physically part of Fhit dimer domains rather than the Nit tetramer (FIG. 3 and FIG. 4). Nearly all of the interactions between Nit and Fhit are mediated by binding of antiparallel NS13 strands to the antiparallel FH1 helices at the bottom of Fhit dimers. The two Nit-Fhit interaction surfaces are extensive, at 1080 Å a piece, but appear more reversible than the east-west interface (7300 Å², including 4900 Å² of Nit-Nit interactions and two 1200 Å² patches of Fhit-Fhit interactions) or the north-south interface (2350 Å²) within the NitFhit tetramer. Consistent with biochemical data (Table 2), the Nit tetramer does not interact with the nucleotide-binding surface of Fhit dimers. In contrast, the Nit tetramer binds Fhit in a manner that presents nucleotide-binding surfaces of Fhit (Pace, H. C., et al. Proc. Natl. Acad. Sci. USA 95, 5484-5489 1998) at the two extreme poles of the complex, potentially for interaction with Fhit effectors.

Plasticity in Nit and a Candidate Nit Active Site

The differences in protein conformation between the northern and southern molecules are largely localized to NH2 (FIG. 5). Ethylmercury bound to Cys75 in the northern but not the southern chains. Without ethylmercury bound, residues 70 through 76 are not helical. Upon binding of ethylmercury, NH2 becomes continuously helical but is bent at residue 75. Though it is not obvious what sequence feature in worm NH2 disrupts its helicity, the NH2 helix is unique among the Nit helices in that homologs have insertions, deletions and Gly and Pro substitutions in this region (FIG. 1). Thus, the disrupted nature of the NH2 helix appears to be a conserved feature that may be functionally important.

Nitrilases are thiol enzymes that the attack the cyano carbon of nitriles (R—C—N) to form a covalent thioimidate complex (Stevenson, D. E., et al., Biotechnology & Applied Biochemistry 15: 283-302, 1992). Addition of one water molecule is accompanied by release of ammonia and transformation of the planar thioimidate to a planar thiol acylenzyme via a tetrahedral intermediate. Addition of a second water molecule would allow the acid product to leave and regenerate the enzyme (Stevenson, D. E., et al., Biotechnology & Applied Biochemistry 15: 283-302, 1992). Similarly, a related family of aliphatic acid amidases uses the conserved cysteine to acylate and release ammonia from acid amides (R—CONH₂) (Novo, C., et al., FEBS Letters 367: 275-279, 1995). Cys 169, which aligns with the conserved Cys of nitrilases and amidases was located on the solvent exposed face of the Nit β-sheet and was modified by ethylmercury (FIG. 6). Only 3.0 Å and 3.7 Å from Cys 169, we located Glu 54 and Lys 127, both conserved in nitrilases. In nitrilases, the corresponding residues could function as a catalytic triad with Glu acting as the general base for the thiol.

Discussion

According to the theory of Rosetta Stone proteins, proteins that engage in fusion events are expected to jointly participate in a biochemical or cellular pathway and/or to physically interact (Marcotte, E. M., et al., Science 285: 751-753, 1999). Nonetheless, the least presumptuous expectation about NitFhit would have been that homotypic Fhit interactions would drive dimerization of NitFhit and that Nit would neither be multimerized nor bound to Fhit. The present invention relates to a stable NitFhit tetramer that displays two Fhit dimers on opposite poles and has extensive homo- and hetero-oligomeric interactions. Strikingly, the most C-terminal β-strand of Nit polypeptide sequences exits the tetrameric Nit domain and binds Fhit dimer domains. Fhit dimer domains are bound with their nucleotide-binding surfaces (Pace, H. C., et al., Proc. Natl. Acad. Sci. 95: 5484-5489, 1998) facing away from Nit. Thus, Nit is unlikely to be a Fhit effector that detects that ApₙA state of Fhit.

It was hypothesized that proteins with low affinity heterotypic interactions that function in the same process might provide a selection for fusion events (Marcotte, E. M., et al., Science 285: 751-753, 1999). Upon fusion, the local concentration of the binding partner is greatly increased from that of separate polypeptides. The structure of NitFhit supports the view that Nit and Fhit are reversibly interacting proteins whose degree of hetero-oligomerization is less than the degree of Fhit dimerization or Nit tetramerization and this is supported by biochemical and two-hybrid assays.

Though substrates for animal Nit proteins have not been identified, the structure of NitFhit is the basis for prediction of a Cys-Glu-Lys catalytic triad in the nitrilase superfamily. The nitrilase from R. rhodocrous J1 has been purified and tested for pH- dependence of benzonitrile hydrolysis. Consistent with Glu elevating the pKa of Cys and functioning as a general base, recombinant R. rhodocrous J1 nitrilase showed no pH-dependence between pH 5.5 and 10.0, which were the limits of the enzyme's physical stability (Milano, S. K, Schimerlik, M., Brenner, C. [In preparation]).

Cancer cells that are Fhit-deficient are defective in programmed cell death (Ji, L., et al., Cancer Research 59:3333-3339, 1999; Sard, L., et al., Proc. Natl. Acad. Sci. 96: 8489-8492, 1999) yet the point of action of Fhit in apoptosis is unclear. Three well-known signals for cell cycle arrest and programmed cell death, namely contact inhibition of growth (Segal, E., Le Pecq, J. B. Exp. Cell Res. 167:119-126, 1986), interferons (Vartanian, A., et al., FEBS Lett. 381: 32-34, 1996), and etoposide (Vartanian, A. FEBS Lett. 415: 160-162, 1997), induce synthesis of diadenosine polyphosphates, the likely positive regulators of the cellular activity of Fhit (Pace, H. C., et al., Proc. Natl. Acad. Sci. USA 95: 5484-5489, 1998). While Fhit is likely to function in an animal cell death pathway, identification of Fhit and Nit proteins in fungi suggests that these proteins have a more fundamental function in maintaining the differentiated states of single cells. By mutating residues in the putative active site of yeast or animal Nit proteins, it will be possible to trap Nit substrates or binding partners and use reverse genetic approaches to discover the cellular consequences of Nit activity, Nit-Fhit heteromultimerization, and the cellular targets of this pathway.

Nit Coding Sequences

Human (H. sapiens) Nit2 (SEQ. ID. NO: 8) and mouse (M. musclulus) Nit2 (SEQ. ID. NO:9) cDNA sequences and sequences complementary thereto; and frog (X. laevis) Nit1 cDNA sequences (SEQ. ID. NO: 10) and sequences complementary thereto are: human (H. sapiens) Nit2, mouse (M. musclulus) Nit2, and frog (X. laevis) Nit1 nucleic acids provided by the present invention. In a specific embodiment herein, a human (H. sapiens) Nit2, mouse (M. musclulus) Nit2, and frog (X. laevis) Nit1 cDNA sequence is provided, thus lacking any introns. Sequences hybridizable thereto, preferably lacking introns, are also provided. Nucleic acids comprising human (H. sapiens) Nit2, mouse (M. musclulus) Nit2, and frog (X. laevis) Nit1 DNA or RNA exon sequences are also provided; in various embodiments, at least 15, 25 or 50 contiguous nucleotides of exon sequences are in the nucleic acid. Also included within the scope of the present invention are nucleic acids comprising human (H. sapiens) Nit2, mouse (M. musclulus) Nit2, and frog (X. laevis) Nit1 cDNA or RNA consisting of at least 8 nucleotides, at least 15 nucleotides, at least 25 nucleotides, at least 50 nucleotides, at least 100 nucleotides, at least 200 nucleotides, or at least 350 nucleotides. In various embodiments, nucleic acids are provided that are less than 2,000, less than 500, less than 275, less than 200, less than 100, or less than 50 bases (or bp, if double-stranded). In various embodiments, the nucleic acids are less than 300 kb, 200 kb, 100 kb, 50 kb, or 10 kb. Nucleic acids can be single-stranded or double-stranded. In specific embodiments, isolated nucleic acids are provided that comprise at least 15 contiguous nucleotides coding sequences but which do not comprise all or a portion of any intron. In a specific embodiment, the nucleic acid comprises at least one coding exon. In yet another specific embodiment, the nucleic acid comprising human (H. sapiens) Nit2, mouse (M. musclulus) Nit2, and frog (X. laevis) Nit1 gene exon sequences does not contain sequences of a genomic flanking gene (i.e., 5′ or 3′ to the Nit2 gene in the genome).

The invention also provides single-stranded oligonucleotides for use as primers in PCR that amplify a human (H. sapiens) Nit2, mouse (M. musclulus) Nit2, and frog (X. laevis) Nit1 sequence-containing fragment, e.g., an oligonucleotide having the sequence of a hybridizable portion (at least 8 nucleotides) of a human (H. sapiens) Nit2, mouse (M. musclulus) Nit2, and frog (X. laevis) Nit1 gene, and another oligonucleotide having the reverse complement of a downstream sequence in the same strand of the human (H. sapiens) Nit2, mouse (M. musclulus) Nit2, and frog (X. laevis) Nit1 gene, such that each oligonucleotide primes synthesis in a direction toward the other. The oligonucleotides are preferably in the range of 10-35 nucleotides in length.

The Nit cDNA sequences for human (H. sapiens) Nit2 (SEQ. ID. NO: 8), mouse (M. musclulus) Nit2 (SEQ. ID. NO:9), and frog (X. laevis) Nit1 (SEQ. ID. NO: 10), are provided in the present invention.

In accordance with the present invention, any polynucleotide sequence which encodes the amino acid sequence of a human (H. sapiens) Nit2, mouse (M. musclulus) Nit2, and frog (X. laevis) Nit1 gene product can be used to generate recombinant molecules which direct the expression of human (H. sapiens) Nit2, mouse (M. musclulus) Nit2, and frog (X. laevis) Nit1. Included within the scope of the present invention are nucleic acids consisting of at least 8 nucleotides that are useful as probes or primers (i.e., a hybridizable portion) in the detection or amplification of human (H. sapiens) Nit2, mouse (M. musclulus) Nit2, and frog (X. laevis) Nit1.

In a specific embodiment disclosed herein, the invention relates to the nucleic acid sequence of the human Nit2 cDNA (SEQ. ID. NO: 8). The present invention also provides nucleic acid sequences of mouse (M. musclulus) Nit2 (SEQ. ID. NO:9), and frog (X. laevis) Nit1 cDNA (SEQ. ID. NO: 10). The invention also relates to nucleic acid sequences hybridizable or complementary to the foregoing sequences or equivalent to the foregoing sequences in that the equivalent nucleic acid sequences also encode a protein product displaying human (H. sapiens) Nit2, mouse (M. musclulus) Nit2, and frog (X. laevis) Nit1) functional activity.

The invention also relates to nucleic acids hybridizable to or complementary to the above-described nucleic acids. In specific aspects, nucleic acids are provided which comprise a sequence complementary to at least 10, 25, 50, 100, or 200 nucleotides or the entire coding region of a human (H. sapiens) Nit2, mouse (M. musclulus) Nit2, and frog (X. laevis) Nit1 gene. In a specific embodiment, a nucleic acid which is hybridizable to a human (H. sapiens) Nit2, mouse (M. musclulus) Nit2, and frog (X. laevis) Nit1 nucleic acid , or to a nucleic acid encoding a derivative thereof, under conditions of low stringency is provided. By way of example and not limitation, procedures using such conditions of low stringency are as follows (see also Shilo, B. Z., Weinberg, R. A. Proc. Natl. Acad. Sci. USA 78:6789-6792, 1981): Filters containing DNA are pretreated for 6 h at 40.degree. C. in a solution containing 35% formamide, 5.times. SSC, 50 mM Tris-HCl (pH 7.5), 5 mM EDTA, 0.1% PVP, 0.1% Ficoll, 1% BSA, and 500 .mu.g/ml denatured salmon sperm DNA. Hybridizations are carried out in the same solution with the following modifications: 0.02% PVP, 0.02% Ficoll, 0.2% BSA, 100 .mu.g/ml salmon sperm DNA, 10% (wt/vol) dextran sulfate, and 5-20.times.10.sup.6 cpm .sup.32 P-labeled probe is used. Filters are incubated in hybridization mixture for 18-20 h at 40.degree. C., and then washed for 1.5 h at 55.degree. C. in a solution containing 2.times. SSC, 25 mM Tris-HCl (pH 7.4), 5 mM EDTA, and 0.1% SDS. The wash solution is replaced with fresh solution and incubated an additional 1.5 h at 60.degree. C. Filters are blotted dry and exposed for autoradiography. If necessary, filters are washed for a third time at 65-68.degree. C. and re-exposed to film. Other conditions of low stringency which may be used are well known in the art (e.g., as employed for cross-species hybridizations).

In another specific embodiment, a nucleic acid which is hybridizable to a human (H. sapiens) Nit2, mouse (M. musclulus) Nit2, and frog (X. laevis) Nit1 nucleic acid under conditions of high stringency is provided (see infra).

In a preferred aspect, polymerase chain reaction (PCR) is used to amplify a desired nucleic acid sequence in a library or from a tissue source by using oligonucleotide primers representing known Nit2, or frog (X. laevis) Nit1 sequences. Such primers may be used to amplify sequences of interest from an RNA or DNA source, preferably a cDNA library. PCR can be carried out, e.g., by use of a Perkin-Elmer Cetus thermal cycler and Taq polymerase (Gene Amp.TM.). The DNA being amplified can include mRNA or cDNA or genomic DNA from any eukaryotic species. One can choose to synthesize several different degenerate primers, for use in the PCR reactions. It is also possible to vary the stringency of hybridization conditions used in priming the PCR reactions, to allow for greater or lesser degrees of nucleotide sequence homology between the Nit2, or frog (X. laevis) Nit1 gene being cloned and the known Nit2, or frog (X. laevis) Nit1 gene. Other means for primer dependent amplification of nucleic acids are known to those of skill in the art and can be used.

After successful amplification of a segment of a Nit2, or frog (X. laevis) Nit1 gene that segment may be molecularly cloned and sequenced, and utilized as a probe to isolate a complete cDNA or genomic clone. This, in turn, will permit the determination of the gene's complete nucleotide sequence, the analysis of its expression, and the production of its protein product for functional analysis. In this fashion, additional genes encoding homologous proteins are identified. Alternatively, the Nit2, or frog (X. laevis) Nit1 gene of the present invention may be isolated through an exon trapping system, using genomic DNA (Nehls, M., et al., Oncogene 9(8): 2169-2175, 1994; Verna, et al., Nucleic Acids Res 21(22):5198:5202, 1993; Auch, D., et al., Nucleic Acids Res 18(22):6743-6744, 1990).

Potentially, any eukaryotic cell can serve as the nucleic acid source for the molecular cloning of the Nit2, or frog (X. laevis) Nit1 gene. The nucleic acid sequences encoding Nit2, or frog (X. laevis) Nit1 can be isolated from, for example, human, porcine, bovine, feline, avian, equine, canine, rodent, as well as additional primate sources. The DNA may be obtained by standard procedures known in the art from, for example, cloned DNA (e.g., a DNA “library”), by chemical synthesis, by cDNA cloning, or by the cloning of genomic DNA, or fragments thereof, purified from a desired cell. (See, for example, Sambrook, et al., 1989, Molecular Cloning, A Laboratory Manual. New York: Laboratory Press, 1985; DNA Cloning: A Practical Approach, Vol. I, II. U.K.: MRL Press, Ltd., Oxford.) The gene should be molecularly cloned into a suitable vector for propagation of the gene.

In the molecular cloning of the gene from genomic DNA, DNA fragments are generated, some of which will encode the desired gene. The DNA may be cleaved at specific sites using various restriction enzymes. Alternatively, one may use DNAse in the presence of manganese to fragment the DNA, or the DNA can be physically sheared, as for example, by sonication. The linear DNA fragments can then be separated according to size by standard techniques, including but not limited to, agarose and polyacrylamide gel electrophoresis and column chromatography.

Once the DNA fragments are generated, identification of the specific DNA fragment containing the desired gene may be accomplished in a number of ways. For example, a Nit2, or frog (X. laevis) Nit1 gene or cDNA of the present invention or its specific RNA, or a fragment thereof, such as a probe or primer, may be isolated and labeled and then used in hybridization assays to detect a generated Nit2, or frog (X. laevis) Nit1 gene, (Benton, W., Davis, R., Science 196:180, 1977; Grunstein, M., Hogness, D., Proc. Natl. Acad. Sci 72:3961, 1975). Those DNA fragments sharing substantial sequence homology to the probe will hybridize under high stringency conditions. The phrase “high stringency conditions” as used herein refers to those hybridizing conditions that (1) employ low ionic strength and high temperature for washing, for example, 0.015 M NaCl/0.0015 M sodium citrate/0.1% SDS at 50.degree. C.; (2) employ during hybridization a denaturing agent such as formamide, for example, 50% (vol/vol) formamide with 0.1% bovine serum albumin/0.1% Ficoll/0.1% polyvinylpyrrolidone/50 mM sodium phosphate buffer at pH 6.5 with 750 mM NaCl, 75 mM sodium citrate at 42.degree. C.; or (3) employ 50% formamide, 5.times. SSC (0.75 M NaCl, 0.075 M sodium pyrophosphate, 5.times. Denhardt's solution, sonicated salmon sperm DNA (50 g/ml), 0.1% SDS, and 10% dextran sulfate at 42.degree. C., with washes at 42.degree. C. in 0.2.times. SSC and 0.1% SDS.

It is also possible to identify the appropriate fragment by restriction enzyme digestion(s) and comparison of fragment sizes with those expected according to a known restriction map. Further selection can be carried out on the basis of the properties of the gene. Alternatively, the presence of the gene may be detected by assays based on the physical, chemical, or immunological properties of its expressed product. For example, cDNA clones, or genomic DNA clones which hybrid-select the proper mRNAs, can be selected which produce a protein that has similar or identical electrophoretic migration, isolectric focusing behavior, proteolytic digestion maps, binding activity or antigenic properties as known for Nit2, or frog (X. laevis) Nit1. Alternatively, the protein may be identified by binding of labeled antibody to the putatively expressing clones, e.g., in an ELISA (enzyme-linked immunosorbent assay)-type procedure.

The Nit2, or frog (X. laevis) Nit1 gene can also be identified by mRNA selection by nucleic acid hybridization followed by in vitro translation. In this procedure, fragments are used to isolate complementary mRNAs by hybridization. Such DNA fragments may represent available, purified DNA of another Nit2, or frog (X. laevis) Nit1 gene. Immunoprecipitation analysis or functional assays of the in vitro translation products of the isolated products of the isolated mRNAs identifies the mRNA and, therefore, the complementary DNA fragments that contain the desired sequences. In addition, specific mRNAs may be selected by adsorption of polysomes isolated from cells to immobilized antibodies specifically directed against Nit2, or frog (X. laevis) Nit1 protein. A radiolabelled Nit2, or frog (X. laevis) Nit1 cDNA can be synthesized using the selected mRNA (from the adsorbed polysomes) as a template. The radiolabelled mRNA or cDNA may then be used as a probe to identify the Nit2, or frog (X. laevis) Nit1 DNA fragments from among other genomic DNA fragments.

Alternatives to isolating the genomic DNA include, but are not limited to, chemically synthesizing the gene sequence itself from a known sequence or making cDNA to the mRNA which encodes the Nit2, or frog (X. laevis) Nit1 protein. For example, RNA useful in cDNA cloning of the gene can be isolated from cells which express Nit2, or frog (X. laevis) Nit1. Other methods are known to those of skill in the art and are within the scope of the invention.

The identified and isolated gene can then be inserted into an appropriate cloning vector. A large number of vector-host systems known in the art may be used. Possible vectors include, but are not limited to, plasmids or modified viruses, but the vector system must be compatible with the host cell used. Such vectors include, but are not limited to, bacteriophages such as lambda derivatives, or plasmids such as PBR322 or pUC plasmid derivatives or the Bluescript vector (Stratagene). The insertion into a cloning vector can, for example, be accomplished by ligating the DNA fragment into a cloning vector which has complementary cohesive termini. However, if the complementary restriction sites used to fragment the DNA are not present in the cloning vector, the ends of the DNA molecules may be enzymatically modified. Alternatively, any site desired may be produced by ligating nucleotide sequences (linkers) onto the DNA termini; these ligated linkers may comprise specific chemically synthesized oligonucleotides encoding restriction endonuclease recognition sequences. In an alternative method, the cleaved vector and gene may be modified by homopolymeric tailing. Recombinant molecules can be introduced into host cells via transformation, transfection, infection, electroporation, or other methods known to those of skill in the art, so that many copies of the gene sequence are generated.

In an alternative method, the desired gene may be identified and isolated after insertion into a suitable cloning vector in a “shot gun” approach. Enrichment for the desired gene, for example, by size fractionization, can be done before insertion into the cloning vector.

In specific embodiments, transformation of host cells with recombinant DNA molecules that incorporate the isolated Nit2, or frog (X. laevis) Nit1 gene, cDNA, or synthesized DNA sequence enables generation of multiple copies of the gene. Thus, the gene may be obtained in large quantities by growing transformants, isolating the recombinant DNA molecules from the transformants and, when necessary, retrieving the inserted gene from the isolated recombinant DNA.

Oligonucleotides containing a portion of the Nit2, or frog (X. laevis) Nit1 coding or non-coding sequences, or which encode a portion of the protein (e.g., primers for use in PCR) can be synthesized by standard methods commonly known in the art. Such oligonucleotides preferably have a size in the range of 8 to 25 nucleotides. In a specific embodiment herein, such oligonucleotides have a size in the range of 15 to 25 nucleotides or 15 to 35 nucleotides.

The Nit2, or frog (X. laevis) Nit1 sequences provided by the instant invention include those nucleotide sequences encoding substantially the same amino acid sequences as found in native proteins, and those encoded amino acid sequences with functionally equivalent amino acids, as well as those encoding other derivatives or analogs.

Generation of Antibiodies

According to the invention, Nit2, budding yeast (S. cerevisae) Nit3, fission yeast (S. pombe) Nit1, or frog (X. laevis) Nit1 proteins, its fragments or other derivatives, or analogs thereof, may be used as an immunogen to generate antibodies which recognize such an immunogen. Such antibodies include but are not limited to polyclonal, monoclonal, chimeric, single chain, Fab fragments, and an Fab expression library. In a specific embodiment, antibodies to a human protein are produced.

Various procedures known in the art may be used for the production of polyclonal and monoclonal antibodies to a Nit2, budding yeast (S. cerevisae) Nit3, fission yeast (S. pombe) Nit1, or frog (X. laevis) Nit1 protein or derivative or analog. Materials and methods for which are described in Harlow, E., Lane, D., Antibody Laboratory Manual, Cold Spring Harbor, 1998, which is incorporated herein by reference.

According to the invention, techniques described for the production of single chain antibodies (U.S. Pat. No. 4,946,778) can be adapted to produce Nit2, budding yeast (S. cerevisae) Nit3, fission yeast (S. pombe) Nit1, or frog (X. laevis) Nit1-specific single chain antibodies. An additional embodiment of the invention utilizes the techniques described for the construction of Fab expression libraries (Huse, et al., Science 246:1275-1281, 1989) to allow rapid and easy identification of monoclonal Fab fragments with the desired specificity for Nit2, budding yeast (S. cerevisae) Nit3, fission yeast (S. pombe) Nit1, or frog (X. laevis) Nit1 proteins, derivatives, or analogs.

Antibody fragments which contain the idiotype of the molecule can be generated by known techniques. For example, such fragments include but are not limited to: the F(ab′).sub.2 fragment which can be produced by pepsin digestion of the antibody molecule; the Fab′ fragments which can be generated by reducing the disulfide bridges of the F(ab).sub.2 fragment, and the Fab fragments which can be generated by treating the antibody molecule with papain and a reducing agent.

In the production of antibodies, screening for the desired antibody can be accomplished by techniques known in the art.

The foregoing antibodies can be used in methods known in the art relating to the localization and activity of the protein sequences of the invention, e.g., for imaging these proteins, measuring levels thereof in appropriate physiological samples, in diagnostic methods, etc.

Proteins, Derivatives and Analogs thereof

The invention further relates to Nit2, budding yeast (S. cerevisae) Nit3, fission yeast (S. pombe) Nit1, or frog (X. laevis) Nit1 proteins, and derivatives (including but not limited to fragments) and analogs thereof. Nucleic acids encoding Nit2, budding yeast (S. cerevisae) Nit3, fission yeast (S. pombe) Nit1, or frog (X. laevis) Nit1 protein derivatives and protein analogs are also provided. Molecules comprising Nit2, budding yeast (S. cerevisae) Nit3, fission yeast (S. pombe) Nit1, or frog (X. laevis) Nit1-proteins or derivatives are also provided.

The production and use of derivatives and analogs related to Nit2, budding yeast (S. cerevisae) Nit3, fission yeast (S. pombe) Nit1, or frog (X. laevis) Nit1 are within the scope of the present invention. In a specific embodiment, the derivative or analog is functionally active, i.e., capable of exhibiting one or more functional activities associated with a full-length, wild-type protein. As one example, such derivatives or analogs which have the desired immunogenicity or antigenicity can be used, for example, in immunoassays, for immunization, for inhibition of Nit2, budding yeast (S. cerevisae) Nit3, fission yeast (S. pombe) Nit1, or frog (X. laevis) Nit1 activity, etc. Derivatives or analogs that retain, or alternatively lack or inhibit, a desired Nit2, budding yeast (S. cerevisae) Nit3, fission yeast (S. pombe) Nit1, or frog (X. laevis) Nit1 property of interest (e.g., inhibition of cell proliferation, tumor inhibition), can be used as inducers, or inhibitors, respectively, of such property and its physiological correlates. A specific embodiment relates to a Nit2, budding yeast (S. cerevisae) Nit3, fission yeast (S. pombe) Nit1, or frog (X. laevis) Nit1 fragment that can be bound by an anti-Nit antibody. Derivatives or analogs of Nit2, budding yeast (S. cerevisae) Nit3, fission yeast (S. pombe) Nit1, or frog (X. laevis) Nit1 can be tested for the desired activity by procedures known in the art.

In particular, derivatives can be made by altering Nit2, budding yeast (S. cerevisae) Nit3, fission yeast (S. pombe) Nit1, or frog (X. laevis) Nit1 sequences by substitutions, additions or deletions that provide for functionally equivalent molecules. Due to the degeneracy of nucleotide coding sequences, other DNA sequences which encode substantially the same amino acid sequence as a Nit2, budding yeast (S. cerevisae) Nit3, fission yeast (S. pombe) Nit1, or frog (X. laevis) Nit1 gene may be used in the practice of the present invention. These include but are not limited to nucleotide sequences comprising all or portions of Nit2, budding yeast (S. cerevisae) Nit3, fission yeast (S. pombe) Nit1, or frog (X. laevis) Nit1 genes which are altered by the substitution of different codons that encode a functionally equivalent amino acid residue within the sequence, thus producing a silent change. Likewise, the derivatives of the invention include, but are not limited to, those containing, as a primary amino acid sequence, all or part of the amino acid sequence of a Nit2, budding yeast (S. cerevisae) Nit3, fission yeast (S. pombe) Nit1, or frog (X. laevis) Nit1 protein including altered sequences in which functionally equivalent amino acid residues are substituted for residues within the sequence resulting in a silent change. For example, one or more amino acid residues within the sequence can be substituted by another amino acid of a similar polarity which acts as a functional equivalent, resulting in a silent alteration.

Substitutes for an amino acid within the sequence may be selected from other members of the class to which the amino acid belongs. For example, the nonpolar (hydrophobic) amino acids include alanine, leucine, isoleucine, valine, proline, phenylalanine, tryptophan and methionine. The polar neutral amino acids include glycine, serine, threonine, cysteine, tyrosine, asparagine, and glutamine. The positively charged (basic) amino acids include arginine, lysine and histidine. The negatively charged (acidic) amino acids include aspartic acid and glutamic acid.

In a specific embodiment of the invention, proteins consisting of or comprising a fragment of a Nit2, budding yeast (S. cerevisae) Nit3, fission yeast (S. pombe) Nit1, or frog (X. laevis) Nit1 protein consisting of at least 10 (continuous) amino acids of the protein is provided. In other embodiments, the fragment consists of at least 20 or 50 amino acids of the Nit2, budding yeast (S. cerevisae) Nit3, fission yeast (S. pombe) Nit1, or frog (X. laevis) Nit1 protein. In specific embodiments, such fragments are not larger than 35, 100 or 140 amino acids. Derivatives or analogs of Nit2, budding yeast (S. cerevisae) Nit3, fission yeast (S. pombe) Nit1, or frog (X. laevis) Nit1 include but are not limited to those molecules comprising regions that are substantially homologous to Nit2, budding yeast (S. cerevisae) Nit3, fission yeast (S. pombe) Nit1, or frog (X. laevis) Nit1 or fragments thereof (e.g., in various embodiments, at least 60% or 70% or 80% or 90% or 95% identity over an amino acid sequence of identical size or when compared to an aligned sequence in which the alignment is done by a computer homology program known in the art) or whose encoding nucleic acid is capable of hybridizing to a coding Nit2, budding yeast (S. cerevisae) Nit3, fission yeast (S. pombe) Nit1, or frog (X. laevis) Nit1 sequence, under stringent, moderately stringent, or nonstringent conditions.

The Nit2, budding yeast (S. cerevisae) Nit3, fission yeast (S. pombe) Nit1, or frog (X. laevis) Nit1 derivatives and analogs of the invention can be produced by various methods known in the art. The manipulations which result in their production can occur at the gene or protein level.

Additionally, the Nit2, budding yeast (S. cerevisae) Nit3, fission yeast (S. pombe) Nit1, or frog (X. laevis) Nit1 encoding nucleic acid sequence can be mutated in vitro or in vivo, to create and/or destroy translation, initiation, and/or termination sequences, or to create variations in coding regions and/or form new restriction endonuclease sites or destroy preexisting ones, to facilitate further in vitro modification. Any technique for mutagenesis known in the art can be used, including but not limited to, chemical mutagenesis, in vitro site-directed mutagenesis (Hutchinson, C., et al., J Biol Chem 253:6551, 1978), etc.

Manipulations of the Nit2, budding yeast (S. cerevisae) Nit3, fission yeast (S. pombe) Nit1, or frog (X. laevis) Nit1 sequence may also be made at the protein level. Included within the scope of the invention are protein fragments or other derivatives or analogs which are differentially modified during or after translation, e.g., by glycosylation, acetylation, phosphorylation, amidation, derivatization by known protecting/blocking groups, proteolytic cleavage, linkage to an antibody molecule or other cellular ligand, etc. Any of numerous chemical modifications may be carried out by known techniques, including but not limited to specific chemical cleavage by cyanogen bromide, trypsin, chymotrypsin, papain, V8 protease, NaBH.sub.4 acetylation, formylation, oxidation, reduction; metabolic synthesis in the presence of tunicamycin; etc.

In addition, analogs and derivatives of Nit2, budding yeast (S. cerevisae) Nit3, fission yeast (S. pombe) Nit1, or frog (X. laevis) Nit1 can be chemically synthesized. For example, a peptide corresponding to a portion of a Nit2, budding yeast (S. cerevisae) Nit3, fission yeast (S. pombe) Nit1, or frog (X. laevis) Nit1 protein which comprises the desired domain, or which mediates the desired activity in vitro, can be synthesized by use of a peptide synthesizer. Furthermore, if desired, nonclassical amino acids or chemical amino acid analogs can be introduced as a substitution or addition into the Fhit sequence. Non-classical amino acids include but are not limited to the D-isomers of the common amino acids, .alpha.-amino isobutyric acid, 4amino-butyric acid, Abu, 2-amino butyric acid, .gamma.-Abu, epsilon.-Ahx, 6-amino hexanoic acid, Aib, 2-amino isobutyric acid, 3-amino propionic acid, ornithine, norleucine, norvaline, hydroxyproline, sarcosine, citrulline, cysteic acid, t-butylglycine, t-butylalanine, phenylglycine, cyclohexylalanine, .beta.-alanine, fluoro-amino acids, designer amino acids such as .beta.-methyl amino acids, C.alpha.-methyl amino acids, N.alpha.-methyl amino acids, and amino acid analogs in general. Furthermore, the amino acid can be D (dextrorotary) or L (levorotary).

In a specific embodiment, the Nit2, budding yeast (S. cerevisae) Nit3, fission yeast (S. pombe) Nit1, or frog (X. laevis) Nit1 derivative is a chimeric, or fusion, protein comprising a Nit2, budding yeast (S. cerevisae) Nit3, fission yeast (S. pombe) Nit1, or frog (X. laevis) Nit1 protein or fragment thereof (preferably consisting of at least a domain or motif of the protein, or at least 10 amino acids of the protein) joined at its amino- or carboxy-terminus via a peptide bond to an amino acid sequence of a different protein. In one embodiment, such a chimeric protein is produced by recombinant expression of a nucleic acid encoding the protein (comprising a Nit2, budding yeast (S. cerevisae) Nit3, fission yeast (S. pombe) Nit1, or frog (X. laevis) Nit1-coding sequence joined in-frame to a coding sequence for a different protein). Such a chimeric product can be made by ligating the appropriate nucleic acid sequences encoding the desired amino acid sequences to each other by methods known in the art, in the proper coding frame, and expressing the chimeric product by methods commonly known in the art. Alternatively, such a chimeric product may be made by protein synthetic techniques, e.g., by use of a peptide synthesizer. Chimeric genes comprising portions of Nit2, budding yeast (S. cerevisae) Nit3, fission yeast (S. pombe) Nit1, or frog (X. laevis) Nit1 fused to any heterologous protein-encoding sequences may be constructed.

In another specific embodiment, the Nit2, budding yeast (S. cerevisae) Nit3, fission yeast (S. pombe) Nit1, or frog (X. laevis) Nit1 derivative is a molecule comprising a region of homology with a Nit2, budding yeast (S. cerevisae) Nit3, fission yeast (S. pombe) Nit1, or frog (X. laevis) Nit1 protein. By way of example, in various embodiments, a first protein region can be considered “homologous” to a second protein region when the amino acid sequence of the first region is at least 30%, 40%, 50%, 60%, 70%, 75%, 80%, 90%, or 95% identical, when compared to any sequence in the second region of an equal number of amino acids as the number contained in the first region or when compared to an aligned sequence of the second region that has been aligned by a computer homology program known in the art. For example, a molecule can comprise one or more regions homologous to a Nit2, budding yeast (S. cerevisae) Nit3, fission yeast (S. pombe) Nit1, or frog (X. laevis) Nit1 domain or a portion thereof or a full-length protein.

Screening for Small Molecules to Regulate Nit2

The present invention relates to the detection of molecules that specifically bind to Nit2 and thereby modify its activity. Such molecules will thus affect cell proliferation. In a preferred embodiment, assays are performed to screen for molecules with potential utility as therapeutic agents or lead compounds for drug development. The invention provides assays to detect molecules that mimic Fhit and induce apoptosis by binding to a Fhit binding site on a Nit2 protein. The invention further provides assays to detect molecules that antagonize formation of a NitFhit hetero-oligomer, thereby inhibiting the activity of Nit and subsequent programmed cell death (apoptosis) while promoting cell proliferation.

For example, recombinant cells expressing Nit2 nucleic acids are used to recombinantly produce Nit2 and screen for molecules that bind to Nit2. Molecules are contacted with the Nit2, or fragment thereof, under conditions conducive to binding, and then molecules that specifically bind to the Nit2 are identified. Methods that are used to carry out the foregoing are commonly known in the art.

In a specific embodiment of the present invention, a Nit2 and/or cell line that expresses a Nit2 is used to screen for antibodies, peptides, or other molecules that bind to the Nit2 and act as a Fhit mimic or antagonist of Fhit. The mimics and antagonists of the present invention will function in any cell. Fhit mimics will activate a Nit2 function, promoting an apoptotic response. Therefore, Fhit mimics of the present invention will inhibit or prevent a disease state associated with excessive cell proliferation. Such disease states include, but are not limited to, leukemias, lymphomas and other cancers, restenosis, etc.

In contrast, Fhit antagonists will modulate the activity of Nit2 and are used to inhibit or prevent a disease state associated with excessive cell death. Such disease states occur in degenerative diseases. For example, Alzheimer's, Armanni-Ehrlich's, macular degenerative diseases, etc.

Fhit mimics and antagonists are identified by screening organic or peptide libraries with recombinantly expressed Nit2. These Fhit mimics and antagonists are useful as therapeutic molecules, or lead compounds for the development of therapeutic molecules, to modify the activity of Nit2. Synthetic and naturally occurring products are screened in a number of ways deemed routine to those of skill in the art.

By way of example, diversity libraries, such as random or combinatorial peptide or nonpeptide libraries are screened for molecules that specifically bind to Nit2. Many libraries are known in the art that are used, e.g., chemically synthesized libraries, recombinant (e.g., phage display libraries), and in vitro translation-based libraries.

Examples of chemically synthesized libraries are described in Fodor, S. P., et al., Science 251:767-773, 1991; Houghten, R. A., et al., Nature 354:84-86, 1991; Lam, K. D., et al., Nature 354:82-84, 1991; Medynski, D. Bio/Technology 12:709-710, 1994; Gallop, M. A., et al., J. Medicinal Chemistry 37(9):1233-1251, 1994; Ohlmeyer, M. H., et al., Proc. Natl. Acad. Sci. USA 90:10922-10926, 1993; Erb, E., et al., Proc. Natl. Acad. Sci. 91:11422-11426, 1994,; Houghten, R. A., et al., Biotechniques 13: 412, 1992; Jayawickreme, C. K., et al., Proc. Natl. Acad. Sci. USA 91:1614-1618, 1994; Salmon, S, E. et al., Proc. Natl. Acad. Sci. USA 90: 11708-11712, 1993; PCT Publication No. WO 93/20242; Brenner, S., Lerner, R. A. Proc. Natl. Acad. Sci. USA 89:5381-5383, 1992.

Examples of phage display libraries are described in Scott J. K., Smith, G. P. Science 249:386-390, 1990; Devlin J. J., et al., Science, 249:404-406, 1990; Christian, R. B., et al., J Mol Biol 227:711-718, 1992; Lenstra, J. A. J. Immunol. Meth. 152:149-157, 1992; Kay, B. K., et al., Gene 128:59-65, 1993; and PCT Publication No. WO 94/18318 dated Aug. 18, 1994.

In vitro translation-based libraries include, but are not limited to, those described in PCT Publication No. WO 91/0505 dated Apr. 18, 1991; and Mattheakis, L. C., et al, Proc. Natl. Acad. Sci. 91: 9022-9026,1994.

By way of examples of nonpeptide libraries, a benzodiazepine library (see e.g., Bunin, B. A., et al., Proc. Natl. Acad. Sci. 91:4708-4712, 1994) can be adapted for use. Peptoid libraries (Simon, R. J. et al., Proc. Natl. Acad. Sci 89:9367-9371, 1992) can also be used. Another example of a library that can be used, in which the amide functionalities in peptides have been permethylated to generate a chemically transformed combinatorial library, is described by Ostresh, J. M., et al., Proc. Natl. Acad. Sci. USA 91: 11138-11142, 1994.

Screening the libraries is accomplished by any of a variety of commonly known methods. See, e.g., the following references, which disclose screening of peptide libraries: Parnley, S. F., Smith, G. P. Adv Exp Med Biol 251:215-218, 1989; Scott, J. K., Smith, G. P. Science 249:386-390, 1990; Fowlkes, D. M., et al., BioTechniques 13:422-427, 1992; Oldenburg, K. R., et al., Proc. Natl. Acad. Sci. USA 89:5393-5397, 1992; Yu et al., Cell 76:933-945, 1994; Staudt L. M. et al., Science 241:577-580, 1988; Bock et al, Nature 355:564-566, 1992; Tuerk C., et al, Proc. Natl. Acad. Sci. USA 89:6988-6992, 1992; Ellington, A. D., et al, Nature 355:850-852, 1992; U.S. Pat. No. 5,096,815, U.S. Pat. No. 5,223,409, and U.S. Pat. No. 5,198,346 all to Ladner et al.; Rebar and Pabo, Science 263:671-673, 1993; and PCT Publication No. WO 94/18318.

In a specific embodiment, screening is carried out by contacting the library members with Nit2, or fragment thereof, immobilized on a solid phase and harvesting those library members that bind to the Nit2, or fragment thereof. Examples of such screening methods, termed “panning” techniques are described by way of example in Parmley, S. F., Smith, G. P. Gene 73:305-318, 1988; Fowlkes, D. M., et al., BioTechniques 13:422-427, 1992; PCT Publication No. WO 94/18318; and in references cited hereinabove.

In another embodiment, the two-hybrid system for selecting interacting proteins in yeast (Fields S., Song, 0. Nature 340:245-246, 1989; Chien, C. T., et al., Proc. Natl. Acad. Sci. USA 88:9578-9582, 1991) is used to identify molecules that specifically bind to Nit2, or fragment thereof.

Therapeutic Uses

The invention provides for treatment or prevention of various diseases and disorders by administration of a therapeutic compound. Such therapeutics include but are not limited to Nit2 proteins and analogs and derivatives (including fragments) thereof; antibodies thereto; nucleic acids encoding the proteins, analogs, or derivatives; and agonists, and antagonists. In a preferred embodiment, disorders involving cell overproliferation are treated or prevented by administration of a therapeutic that promotes Nit2 function.

Generally, administration of products of a species origin or species reactivity (in the case of antibodies) that is the same species as that of the patient is preferred. Thus, in a preferred embodiment, a human Nit2 protein, derivative, or analog, or nucleic acid, or an antibody to a human Nit2 protein or human Nit2 nucleic acid, is therapeutically or prophylactically administered to a human patient.

A Nit2 polynucleotide and its protein product can be used for therapeutic/prophylactic purposes for diseases involving cell overproliferation, as well as other disorders associated with chromosomal translocations or inversions or molecular abnormalities associated with the Nit2 locus, and/or decreased expression of wild-type RNA or protein and/or expression of a mutant RNA or protein. A Nit2 polynucleotide, and its protein product, may be used for therapeutic/prophylactic purposes alone or in combination with other therapeutics useful in the treatment of cancer and hyperproliferative or dysproliferative disorders.

In specific embodiments, therapeutics that promote Nit2 function are administered therapeutically (including prophylactically): (1) in diseases or disorders involving an absence or decreased (relative to normal or desired) level of functional protein, for example, in patients where protein is lacking, genetically defective, biologically inactive or underactive, or underexpressed; or (2) in diseases or disorders wherein in vitro (or in vivo) assays indicate the utility of Nit2 agonist administration. The absence or decreased level in Nit2 protein or function can be readily detected, e.g., by obtaining a patient tissue sample (e.g., from biopsy tissue) and assaying it in vitro for RNA or protein levels, structure and/or activity of the expressed RNA or protein. Many methods standard in the art can be thus employed, including but not limited to immunoassays to detect and/or visualize Nit2 protein (e.g., Western blot, immunoprecipitation followed by sodium dodecyl sulfate polyacrylamide gel electrophoresis, immunocytochemistry, etc.) and/or hybridization assays to detect Nit2 expression by detecting and/or visualizing mRNA or cDNA (e.g., Northern assays, dot blots, in situ hybridization, and preferably those assays), etc.

Therapeutic/Prophylactic Methods and Compositions

The invention provides methods of treatment and prophylaxis by administration to a subject an effective amount of a therapeutic, i.e., a monoclonal (or polyclonal) antibody, retroviral vector, Fhit mimic or Fhit antagonist of the present invention. In a preferred aspect, the therapeutic is substantially purified. The subject is preferably an animal, including but not limited to, animals such as cows, pigs, chickens, etc., and is preferably a mammal, and most preferably human.

Various delivery systems are known and are used to administer a therapeutic of the invention, e.g., encapsulation in liposomes, microparticles, microcapsules, expression by recombinant cells, receptor-mediated endocytosis (see, e.g., Wu, Wu, J Biol Chem 262:4429-4432, 1987), construction of a therapeutic nucleic acid as part of a retroviral or other vector, etc. Methods of introduction include, but are not limited to, intradermal, intramuscular, intraperitoneal, intravenous, subcutaneous, intranasal, and oral routes. The compounds are administered by any convenient route, for example by infusion or bolus injection, by absorption through epithelial or mucocutaneous linings (e.g., oral mucosa, rectal and intestinal mucosa, etc.) and may be administered together with other biologically active agents. Administration can be systemic or local. In addition, it may be desirable to introduce the pharmaceutical compositions of the invention into the central nervous system by any suitable route, including intraventricular and intrathecal injection; intraventricular injection may be facilitated by an intraventricular catheter, for example, attached to a reservoir, such as an Ommaya reservoir.

In a specific embodiment, it may be desirable to administer the pharmaceutical compositions of the invention locally to the area in need of treatment; this may be achieved by, for example, and not by way of limitation, local infusion during surgery, topical application, e.g., in conjunction with a wound dressing after surgery, by injection, by means of a catheter, by means of a suppository, or by means of an implant, the implant being of a porous, non-porous, or gelatinous material, including membranes, such as sialastic membranes, or fibers. In one embodiment, administration is by direct injection at the site (or former site) of a malignant tumor or neoplastic or pre-neoplastic tissue.

In a specific embodiment where the therapeutic is a nucleic acid encoding a protein therapeutic the nucleic acid is administered in vivo to promote expression of its encoded protein, by constructing it as part of an appropriate nucleic acid expression vector and administering it so that it becomes intracellular, e.g., by use of a retroviral vector (see U.S. Pat. No. 4,980,286), or by direct injection, or by use of microparticle bombardment (e.g., a gene gun; Biolistic, Dupont), or coating with lipids or cell-surface receptors or transfecting agents, or by administering it in linkage to a homeobox-like peptide which is known to enter the nucleus (see e.g., Joliot, A. et al., Proc. Natl. Acad. Sci. USA 88:1864-1868, 1991), etc. Alternatively, a nucleic acid therapeutic can be introduced intracellularly and incorporated within host cell DNA for expression, by homologous recombination.

The present invention also provides pharmaceutical compositions. Such compositions comprise a therapeutically effective amount of a therapeutic, and a pharmaceutically acceptable carrier or excipient. Such a carrier includes, but is not limited to, saline, buffered saline, dextrose, water, glycerol, ethanol, and combinations thereof. The carrier and composition can be sterile. The formulation will suit the mode of administration.

The composition, if desired, can also contain minor amounts of wetting or emulsifying agents, or pH buffering agents. The composition can be a liquid solution, suspension, emulsion, tablet, pill, capsule, sustained release formulation, or powder. The composition can be formulated as a suppository, with traditional binders and carriers such as triglycerides. Oral formulation can include standard carriers such as pharmaceutical grades of mannitol, lactose, starch, magnesium stearate, sodium saccharine, cellulose, magnesium carbonate, etc.

In a preferred embodiment, the composition is formulated in accordance with routine procedures as a pharmaceutical composition adapted for intravenous administration to human beings. Typically, compositions for intravenous administration are solutions in sterile isotonic aqueous buffer. Where necessary, the composition also includes a solubilizing agent and a local anesthetic such as lignocaine to ease pain at the site of the injection. Generally, the ingredients are supplied either separately or mixed together in unit dosage form, for example, as a dry lyophilized powder or water free concentrate in a hermetically sealed container such as an ampoule or sachette indicating the quantity of active agent. Where the composition is to be administered by infusion, it is be dispensed with an infusion bottle containing sterile pharmaceutical grade water or saline. Where the composition is administered by injection, an ampoule of sterile water for injection or saline is provided so that the ingredients are mixed prior to administration.

The therapeutics of the invention are formulated as neutral or salt forms. Pharmaceutically acceptable salts include those formed with free amino groups such as those derived from hydrochloric, phosphoric, acetic, oxalic, tartaric acids, etc., and those formed with free carboxyl groups such as those derived from sodium, potassium, ammonium, calcium, ferric hydroxides, isopropylamine, triethylamine, 2-ethylamino ethanol, histidine, procaine, etc.

The amount of the therapeutic of the invention which will be effective in the treatment of a particular disorder or condition will depend on the nature of the disorder or condition, and is determined by standard clinical techniques. In addition, in vitro assays may optionally be employed to help identify optimal dosage ranges. The precise dose to be employed in the formulation will also depend on the route of administration, and the seriousness of the disease or disorder, and is decided according to the judgment of the practitioner and each patient's circumstances. However, suitable dosage ranges for intravenous administration are generally about 20-500 micrograms of active compound per kilogram body weight. Suitable dosage ranges for intranasal administration are generally about 0.01 pg/kg body weight to 1 mg/kg body weight. Effective doses may be extrapolated from dose-response curves derived from in vitro or animal model test systems.

Suppositories generally contain active ingredient in the range of 0.5% to 10k by weight; oral formulations preferably contain 10% to 95% active ingredient.

The invention also provides a pharmaceutical pack or kit comprising one or more containers filled with one or more of the ingredients of the pharmaceutical compositions of the invention. Optionally associated with such container(s) is a notice in the form prescribed by a governmental agency regulating the manufacture, use or sale of pharmaceuticals or biological products, which notice reflects approval by the agency of manufacture, use or sale for human administration.

What is claimed is:

1. An isolated nucleic acid, comprising a nucleotide sequence encoding a human Nit2 protein, wherein the nucleotide sequence is a cDNA sequence.

2. The isolated nucleic acid of claim 1, wherein said nucleotide sequence encodes a human Nit2 protein having an amino acid sequence of SEQ. ID. NO: 1.

3. The isolated nucleic acid of claim 1, comprising a nucleotide sequence of SEQ. ID. NO: 8.

4. A human Nit2 protein.

5. The isolated protein of claim 4, comprising an amino acid sequence of SEQ. ID. NO: 1.

6. An antibody which specifically binds to an epitope of a human Nit2 protein.

7. An isolated nucleic acid, comprising a nucleotide sequence encoding a mouse Nit2 protein, wherein the nucleotide sequence is a cDNA sequence.

8. The isolated nucleic acid of claim 7, wherein said nucleotide sequence encodes a mouse Nit2 protein having an amino acid sequence of SEQ. ID. NO: 2.

9. The isolated nucleic acid of claim 7, comprising a nucleotide sequence of SEQ. ID. NO:9.

10. A mouse Nit2 protein.

11. The isolated protein of claim 10, comprising an amino acid sequence of SEQ. ID. NO: 2.

12. An antibody which specifically binds to an epitope of a mouse Nit2 protein.

13. An S. pombe Nit2 protein.

14. The isolated protein of claim 13, comprising an amino acid sequence of SEQ. ID. NO: 7.

15. An antibody which specifically binds to an epitope of a S. pombe Nit2 protein.

16. An S. cerevisiae Nit3 protein.

17. The isolated protein of claim 16, comprising an amino acid sequence of SEQ. ID. NO: 5.

18. An antibody which specifically binds to an epitope of a S. cerevisae Nit3 protein.

19. An isolated nucleic acid, comprising a nucleotide sequence encoding a X.laevis Nit1 protein, wherein the nucleotide sequence is a cDNA sequence.

20. The isolated nucleic acid of claim 19, wherein said nucleotide sequence encodes a X. laevis Nit1 protein having an amino acid sequence of SEQ. ID. NO: 3.

21. The isolated nucleic acid of claim 19, comprising a nucleotide sequence of SEQ. ID. NO: 10.

22. A X. laevis Nit1 protein.

23. The isolated protein of claim 22, comprising an amino acid sequence of SEQ. ID. NO: 3.

24. An antibody which specifically binds to an epitope of a X. laevis Nit1 protein.

25. A S. pombe Nit1 protein.

26. The isolated protein of claim 25, comprising an amino acid sequence of SEQ. ID. NO: 6.

27. An antibody which specifically binds to an epitope of a S. pombe Nit1 protein.

28. An S. cerevisiae Nit2 protein.

29. The isolated protein of claim 28, comprising an amino acid sequence of SEQ. ID. NO: 4.

30. An antibody which specifically binds to an epitope of a S. cerevisiae Nit2 protein.

31. A method of identifying a molecule that specifically binds to a Nit2 protein and is functionally active in mimicking a Fhit interaction, comprising
a) contacting said Nit2 with a plurality of molecules under conditions conducive to binding between said Nit2 and said molecules; and
b) identifying a molecule within said plurality of molecules that specifically binds to said Nit2 and is functionally active in mimicking said Fhit interaction.

32. A compound comprising a Fhit mimic, wherein said mimic binds to a Nit2 protein in any cell and is functionally active in mimicking a Fhit interaction.

33. A method of treating a disease state in which an activity of a Nit2 protein is altered in a mammal, comprising administering a therapeutically effective amount of a Fhit mimic, wherein said Fhit mimic binds to said Nit2 protein, thereby inducing programmed cell death.

34. The method of claim 33, wherein said disease comprises a proliferative disorder.

35. A pharmaceutical composition comprising a Fhit mimic.

36. A method of identifying a molecule that specifically binds to a Nit2 protein and is functionally active in antagonizing a Fhit interaction, comprising
a) contacting said Nit2 with a plurality of molecules under conditions conducive to binding between said Nit2 and said molecules; and
b) identifying a molecule within said plurality of molecules that specifically binds to said Nit2 and is functionally active in antagonizing said Fhit interaction.

37. A compound comprising a Fhit antagonist, wherein said antagonist binds to a Nit2 protein in any cell and is functionally active in antagonizing a Fhit interaction.

38. A method of treating a disease state in which an activity of a Nit2 protein is altered in a mammal, comprising administering a therapeutically effective amount of a Fhit antagonist, wherein said Fhit antagonist binds to said Nit2 protein, thereby promoting cell proliferation.

39. The method of claim 38, wherein said disease comprises a degenerative disease.

40. A pharmaceutical composition comprising a Fhit antagonist.


SEQUENCE LISTING (11 sequences)

SEQ ID NO: 1 - protein, 276 aa, Homo sapien
MTSFRLALIQLQISSIKSDNVTRACSFIREAATQGAKIVSLPECFNSPYGAKYFPEYAEKIPGESTQKLSEVAKECSIYLIGGSIPEEDAGKLYNTCAVFGPDGTLLAKYRKIHLFDIDVPGKITFQESKTLSPGDSFSTFDTPYCRVGLGICYDMRFAELAQIYAQRGCQLLVYPGAFNLTTGPAHWELLQRSRAVDNQVYVATASPARDDKASYVAWGHSTVVNPWGEVLAKAGTEEAIVYSDIDLKKLAEIRQQIPVFRQKRSDLYAVEMKKP

SEQ ID NO: 2 - protein, 276 aa, mouse
MSTFRLALIQLQVSSIKSDNLTRACSLVREAAKQGANIVSLPECFNSPYGTTYFPDYAEKIPGESTQKLSEVAKESSIYLIGGSIPEEDAGKLYNTCSVFGPDGSLLVKHRKIHLFDIDVPGKITFQESKTLSPGDSFSTFDTPYCKVGLGICYDMRFAELAQIYAQRGCQLLVYPGAFNLTTGPAHWELLQRARAVDNQVYVATASPARDDKASYVAWGHSTVVDPWGQVLTKAGTEETILYSDIDLKKLAEIRQQIPILKQKRADLYTVESKKP

SEQ ID NO: 3 - protein, 288 aa, X. laevis
MAGAHKPLIAVCQMTSTSDKEKNFATCSRLIREAAGRRACMVFLPEAFDYIGGSIEETLSLAESLHGDTIQRYTQLARECGLWLSLGGFHEKGPNWDTDQRISNSHVVVDNTGHIVSVYRKAHLFDVDLQNGVSLRESSSTLPGAELIRPITSPAGKIGLGVCYDLRFPEFSLALAQQGAELLTYPSAFTLTTGLAHWEVLLRARAIETQCYVVAAAQTDRHNEKRTSYGHAMVVDPWGLVIGQCQEGTGICYAEIDIPYMERVRRDMPVWRHRRTDLYGKISFNKPD

SEQ ID NO: 4 - protein, 307 aa, S. cerevisiae
MTSKLKRVAVAQLCSSADLTKNLKVVKELISEAIQKKADVVFLPEASDYLSQNPLHSRYLAQKSPKFIRQLQSSITDLVRDNSRNIDVSIGVHLPPSEQDLLEGNDRVRNVLLYIDHEGKILQEYQKLHLFDVDVPNGPILKESKSVQPGKAIPDIIESPLGKLGSAICYDIRFPEFSLKLRSMGAEILCFPSAFTIKTGEAHWELLGRARAVDTQCYVLMPGQVGMHDLSDPEWEKQSHMSALEKSSRRESWGHSMVIDPWGKIIAHADPSTVGPQLILADLDRELLQEIRNKMPLWNQRRDDLFH

SEQ ID NO: 5 - protein, 291 aa, S. cerevisiae
MSASKILSQKIKVALVQLSGSSPDKMANLQRAATFIERAMKEQPDTKLVVLPECFNSPYSTDQFRKYSEVINPKEPSTSVQFLSNLANKFKIILVGGTIPELDPKTDKIYNTSIIFNEDGKLIDKHRKVHLFDVDIPNGISFHESETLSPGEKSTTIDTKYGKFGVGICYDMRFPELAMLSARKGAFAMIYPSAFNTVTGPLHWHLLARSRAVDNQVYVMLCSPARNLQSSYHAYGHSIVVDPRGKIVAEAGEGEEIIYAELDPEVIESFRQAVPLTKQRRFDVYSDVNAH

SEQ ID NO: 6 - protein, 276 aa, S. pombe
MTLAAVAQLNSSGSILKNLAICKELISQAAAKGAKCIFFPEASDFIAHNSDEAIELTNHPDCSKFIRDVRESATKHSIFVNICVHEPSKVKNKLLNSSLFIEPLHGEIISRYSKAHLFDVEIKNGPTLKESNTTLRGEAILPPCKTPLGKVGSAICFDIRFPEQAIKLRNMGAHIITYPSAFTEKTGAAHWEVLLRARALDSQCYVIAPAQGGKHNEKRASYGHSMIVDPWGTVIAQYSDISSPNGLIFADLDLNLVDHVRTYIPLLRRNDLYPTI

SEQ ID NO: 7 - protein, 322 aa, S. pombe
MNSKFFGLVQKGTRSFFPSLNFCYTRNIMSVSASSLVPKDFRAFRIGLVQLANTKDKSENLQLARLKVLEAAKNGSNVIVLPEIFNSPYGTGYFNQYAEPIEESSPSYQALSSMAKDTKTYLFGGSIPERKDGKLYNTAMVFDPSGKLIAVHRKIHLFDIDIPGGVSFRESDSLSPGDAMTMVDTEYGKFGLGICYDIRFPELAMIAARNGCSVMIYPGAFNLSTGPLHWELLARARAVDNEMFVACCAPARDMNADYHSWGHSTVVDPFGKVIATTDEKPSIVYADIDPSVMSTARNSVPIYTQRRFDVYSEVLPALKKEE

SEQ ID NO: 8 - DNA, 1359 bp, Homo sapien
  misc_feature (1270)...(1270): n = a, c, g, or t
gtggtgcttgtctgcagagtcatgacctctttccgcttggccctcatccagcttcagatttcttccatcaaatcagataacgtcactcgcgcttgtagcttcatccgggaggcagcaacgcaaggagccaaaatagtttctttgccggaatgctttaattctccatatggagcgaaatattttcctgaatatgcagagaaaattcctggtgaatccacacagaagctttctgaagtagcaaaggaatgcagcatatatctcattggaggctctatccctgaagaggatgctgggaaattatataacacctgtgctgtgtttgggcctgatggaactttactagcaaagtatagaaagatccatctgtttgacattgatgttcctggaaaaattacatttcaagaatctaaaacattgagtccgggtgatagtttctccacatttgatactccttactgcagagtgggtctgggcatctgctacgacatgcggtttgcagagcttgcacaaatctacgcacagagaggctgccagctgttggtatatccaggagcttttaatctgaccactggaccagcccattgggagttacttcagcgaagccgggctgttgataatcaggtgtatgtggccacagcctctcctgcccgggatgacaaagcctcctatgttgcctggggacacagcaccgtggtgaacccttggggggaggttctagccaaagctggcacagaagaagcaatcgtgtattcagacatagacctgaagaagctggctgaaatacgccagcaaatccccgtttttagacagaagcgatcagacctctatgctgtggagatgaaaaagccctaaagtttatgtttctaatgtgtcacagaataggacgatatgattctacaacataatcaactccctattaaattctttaatgaagaaaaaaaatttaaaaaaaaaaaaaaaaacctaggttctctattgagatgagaaagcctcattatgctgacattttccacgccacattaaatagttaaaaaggatgcagcctggagccagagagcagaaagctgggctggttctgaagcttcttccatacttaagttgcctccaagcagtttgtgaaagtatcagatcttggtatcctggtgattgattcacctaatataaatatatttgtgtcatgaacctcttaaaaagttgctgggagttgtaatctccatcatctaggaaaacgtgggtctgggtgctattcttttccaagcaggtaccttgnaagttccatttttgggttcatgagtagctataggaacgcaagggtgatacatctttgggtgttttgccagagaagttgggcagccccac

SEQ ID NO: 9 - DNA, 1292 bp, mouse
gactggcaggagtccctgtggcctgcggtgcaccgtggaagagccatgtctactttccgcctggccctcatacagcttcaagtttcttccattaaatcagataaccttacccgggcttgtagcctagtgcgggaggcagcaaagcaaggtgccaacatagtttctctgcctgagtgcttcaattctccatatggaacaacctactttcctgactatgcagagaagattcctggagagtccacacaaaagctttctgaagtagcaaaggagagcagcatatatctcattggaggctccatccctgaagaggatgctgggaaactgtataatacctgctctgtgtttgggcctgatggaagtttactggtaaagcacaggaagatccatctgtttgacattgatgttcctgggaaaattacgtttcaagaatctaaaacattgagccctggtgatagtttctccacatttgatacgccttactgcaaagtgggcctgggcatctgctatgatatgcgcttcgcggagcttgcacaaatctatgcacaaagaggctgccagctcttggtgtatcctggagctttcaatctgaccacaggaccagcccactgggagctgcttcagcgagcccgggctgttgataatcaggtgtatgtggctacagcctctcctgctcgggatgacaaagcctcgtatgtggcctggggacacagcactgttgtggatccttgggggcaggtcctaaccaaagctggcacggaggaaacaatcctgtactcagacatagacctgaagaagctggctgaaattcggcagcaaatccccattttaaaacagaaacgagcagacctctatacagtggaatcaaagaagccttgatatctgtttcaaaaatgtcaccaacaggatgatgctctgtcagatgatcaactctactacatctcttttttttggagggaggggggaacagggccatttcatgttaattctatcaatgatctgtgccacaaggtcccctattttaattaaaagtttcatctttaattaaaatgtgcttggtaacaatgttctagctcttaactagtctgatggttcctaggcatttcagtcccaagatccttttgaacaattaaaaactgaagcctctaagcattgtttccatgtgtggtgggctggtcccatctgtctgagaaaatgtacatttaccagaacactaattttcatggtgctaatatcccatcaacatgacacttttaaaactttttattaaaaattgttttcatacaataaaaaaa

SEQ ID NO: 10 - DNA, 1214 bp, X. laevis
  misc_feature (1083)...(1083): n = a, c, g or t
gtgagtgacgtgtgcgcagtggcaactaaggcctcctgggaaaatgtagaggagcgtgagcttcgcgggacaggacagggtcttaggctctgccttgtgtccacacgcccttgtgcagactgctatagactgtgactttaaccctgtgtccggatataggggttagaagcctgagtgcaatggctggtgcccacaagcccctgattgccgtgtgccagatgacttcaacctctgataaggagaagaatttcgccacgtgttcgcggctgatccgggaggctgcggggcgtcgcgcttgcatggtgtttctgccggaagcctttgactatatcgggggcagcattgaggagacgctgagtctggctgagtctctacatggggacaccattcagcgttacacccaactcgccagggagtgtgggctctggctttccctggggggatttcatgagaaaggacccaactgggacacggaccaacgcatttccaattctcacgtggttgtggacaacacagggcacatagtatcggtgtaccgcaaggctcacctgtttgacgtagacttgcagaatggagtgtcactcagagagagcagttccaccctccccggagcagagcttattcgccccatcacttctccagcaggaaagattggcctgggggtgtgttacgacctccgcttcccagaattctccttggctctggcccaacaaggagcagaacttctcacttacccttctgccttcaccctcactactggtctggcacattgggaggtgttgctgagagcccgtgccatagaaacccagtgctacgtagttgcagcggcacagacagacagacacaatgagaagaggacgtcctatggtcacgctatggtggtagacccgtgggggctggtcattggccaatgccaggaaggaacaggaatatgttatgctgagattgacattccctacatggagcgtgtgaggcgggacatgccggtgtggaggcaccgcaggactgatctgtatgggaaaatctcctttaataaacccgactgactccataatggatcacctgcacctatgggggcaaagnctttcccctgattgctgaaattcctcaatctgtgactgtgaatgacaatgaacgtgacttggaattgcctggttatggcaccggcaatgattctctacagtaattctcaataaaagtgctgaaccttaaaa

SEQ ID NO: 11 - protein, 346 aa, A. thaliana
MSSTKDMSTVQNATPFNGVAPSTTVRVTIVQSSTVYNDTPATIDKAEKYIVEAASKGAELVLFPEGFIGGYPRGFRFGLAVGVHNEEGRDEFRKYHASAIHVPGPEVARLADVARKNHVYLVMGAIEKEGYTLYCTVLFFSPQGQFLGKHRKLMPTSLERCIWGQGDGSTIPVYDTPIGKLGAAICWENRMPLYRTALYAKGIELYCAPTADGSKEWQSSMLHIAIEGGCFVLSACQFCQRKHFPDHPDYLFTDWYDDKEHDSIVSQGGSVIISPLGQVLAGPNFESEGLVTADIDLGDIARAKLYFDSVGHYSRPDVLHLTVNEHPRKSVTFVTKVEKAEDDSNK